(19) United States
(12) Patent Application Publication
(10) Pub. No.: US 2016/0130199 A1
(43) Pub. Date: May 12, 2016

(54) ZIF-8-90 METAL ORGANIC FRAMEWORK (MOF) MEMBRANES FOR n-BUTANE/i-BUTANE SEPARATIONS

(71) Applicants: Phillips 66 Company, Houston, TX (US); Georgia Tech Research Corporation, Atlanta, GA (US)

(72) Inventors: Sankar Nair, Atlanta, GA (US); Kiwon Eum, Atlanta, GA (US); Fereshteh Rashidi, Atlanta, GA (US); Christopher W. Jones, Atlanta, GA (US); Jeffrey H. Drese, Bartlesville, OK (US)

(73) Assignees: Phillips 66 Company, Houston, TX (US); Georgia Tech Research Corporation, Atlanta, GA (US)

(21) Appl. No.: 14/926,395

(22) Filed: Oct. 29, 2015

Related U.S. Application Data
(60) Provisional application No. 62/076,228, filed on Nov. 6, 2014.

(51) Int. Cl. C07C 7/144; C07F 15/06; C07F 3/06

(52) U.S. Cl. CPC C07C 7/144; C07F 3/06; C07F 15/065

(57) ABSTRACT

A method for forming a hybrid zeolitic imidazolate framework (ZIF) comprises the formation steps of: preparing a first solution comprising: a 2-methylimidazolate or a functionalized derivative thereof; and a carboxaldehyde-2-imidazolate or a functionalized derivative thereof; preparing a second solution comprising a metal ion; and combining the first solution and the second solution to form the hybrid ZIF, wherein a first fraction of 2-methylimidazolate or a functionalized derivative thereof in the hybrid ZIF is from about 5 to about 95 or any value there between and a second fraction carboxaldehyde-2-imidazolate or a functionalized derivative thereof in the hybrid ZIF is 100—the first fraction is disclosed. A metal-organic framework (MOF) comprising the hybrid ZIF and a molecular sieve device comprising the hybrid ZIF are also disclosed.

### PRIOR RELATED APPLICATIONS

This application claims benefit of U.S. Provisional Patent Application Ser. No. 62/076,228, filed on Nov. 6, 2014 for “ZIF-8-90 Metal Organic Framework (MOF) Membranes for n-Butane/i-Butane Separations.”

### FEDERALLY SPONSORED RESEARCH STATEMENT

N/A

### REFERENCE TO MICROFICHE APPENDIX

N/A

### FIELD OF INVENTION

The invention relates to synthesis and characterization of metal-organic framework (MOF) membranes, and, in particular, to a hybrid MOF system, namely a zeolitic imidazolate framework (ZIF)-8-90 mixed-linker material, comprising a mixture of linkers from pure MOF phases ZIF-8 and ZIF-90. The invention also relates to using these mixed-linker ZIF-8-90 materials for n-butane/i-butane separations.

### BACKGROUND OF THE INVENTION

Metal organic frameworks (MOFs) are nanoporous materials consisting of organic linkers coordinated to metal ions in crystalline structures. They are potentially attractive as energy-efficient gas separation materials and membranes. MOFs can be used for separations by exploiting differences in molecular adsorption strength, diffusivity, or both. The vast range of MOF structures and the relative simplicity of their synthesis (in relation to other nanoporous materials like zeolites) creates the possibility of rational design, synthesis, and modification of MOF structures for specific separations. A number of reports have considered the application of MOFs for adsorptive separations,¹⁻³ as well as diffusion-based membrane separations.⁴⁻⁷

A subclass of MOFs, known as zeolitic imidazolate frameworks (ZIFs), consist of metal (mainly tetrahedral Zn²) bridged by the nitrogen atoms of imidazolate linkers. ZIFs form structural topologies equivalent to those found in zeolites and other inorganic nanoporous oxide materials. In the past decade, more than 100 ZIF structures have been synthesized, including crystal topologies not yet realized in zeolites.⁸⁻¹² Several ZIFs are known to have good thermal and chemical stability, high microporosity, and high internal surface area.¹³ ZIFs have created substantial interest for potential use in both diffusive as well as adsorptive separation processes. For example, ZIF-8 is useful for membrane-based separation of hydrogen from hydrocarbons and propylene from propane to potentially replace or debottleneck energy-intensive cryogenic distillation processes. A considerable body of work has appeared on the quantification of molecular diffusion properties of ZIFs (most notably ZIF-8) and their use in membranes for diffusion-dominated separations. It has been shown that molecules with significantly higher kinetic diameters than the nominal pore limiting diameter of ZIF-8 (3.4 Å) can diffuse through its micropores. The diffusivities of a wide range of small gas, hydrocarbon, and oxygenated organic molecules have been measured in ZIF-8. Molecular modeling and experimental measurements have shown that ZIF-8 has high diffusion selectivity for methanol over ethanol, whereas ZIF-90 has moderate selectivity for the same separation.¹⁴ ZIFs have also been studied for adsorptive separations. Recent works have demonstrated the high hydrophobicity of ZIF-8 via adsorption studies of water and a number of liquid organic adsorbates. ZIF-8 has also been identified as a candidate for adsorptive recovery of ethanol, propanol and butanol from water due to its hydrophobicity.¹⁵⁻¹⁶

However, single-linker ZIF materials can only allow “discrete” changes in pore size and adsorption characteristics by variation of the imidazolate linker. Diffusion-based molecular separations are extremely sensitive to small (<0.1 Å) changes in the effective pore size. Only limited diffusive separations are possible with single-linker ZIFs, and de novo design of ZIFs may be required for each new separation target. Similarly, adsorptive separations are sensitive to small changes in the hydrophilicity or organophilicity of the ZIF, which are difficult to design de novo. In previous work, we demonstrated a synthetic approach for a series of mixed-linker ZIF-8-90 and ZIF-7-8 materials by inclusion of 2-carboxyimidazole (ZIF-90 linker) and benzimidazole (ZIF-7 linker) along with 2-methylimidazole (ZIF-8 linker) during synthesis.¹³′¹⁷ Preliminary characterizations revealed these materials had a continuously tunable effective pore size (as measured by nitrogen physisorption) that is between the pore sizes of the single-linker “parent” materials (ZIF-7, ZIF-8, and ZIF-90). We denote the mixed-linker ZIF-8-90 materials as ZIF-8ₓ-90₁₀₋ₓ (0≦x≦100), where x is the percentage of ZIF-8 linkers in the framework.

Accordingly, a mixed-linker ZIF, containing two types of linkers, for example, 2-methylimidazole and carboxyaldehyde-2-imidizole, in two different proportions to allow continuously tunable adsorption and diffusion behavior is needed.

### SUMMARY OF THE INVENTION

The invention relates to synthesis and characterization of metal-organic framework (MOF) membranes, and, in particular, to a hybrid MOF system, namely a zeolitic imidazolate framework (ZIF)-8-90 mixed-linker material, comprising a mixture of linkers from pure MOF phases ZIF-8 and ZIF-90. The invention also relates to using these mixed-linker ZIF-8-90 materials for n-butane/i-butane separations.

The present invention shows that mixed-linker ZIFs, containing two types of linkers, for example, 2-methylimidazole and carboxyaldehyde-2-imidazole, in different proportions, allow continuously tunable adsorption and diffusion behavior. The inventors illustrate this highly tunable behavior by measurements of adsorption and diffusion of hydrocarbons (specifically, n-butane and i-butane), alcohols (methanol, ethanol, and n-butanol), and water in mixed-linker ZIF-8ₓ-90₁₀₀₋ₓ (0<x<100) materials. This work is facilitated by the synthesis of mixed-linker ZIF-8-90 crystals with variable fractions of 2-methylimidazole and 2-carboxyimidazole linkers and with a large range of sizes (from 338 nm to 112 μm), and multiple crystal characterization methods including dynamic light scattering, optical microscopy, NMR, powder FT-Raman spectroscopy, and micro-Raman spectroscopy. Volumetric uptake, gravimetric uptake, and PFG-NMR methods were then used to measure intracrystalline adsorption and diffusion properties of the hydrocarbon, alcohol, and water molecules. The inventors have shown that variation of the mixed-linker fraction (x) allows continuous control of n-butane, i-butane, and n-butanol diffusivities over 2-3 orders of magnitude, as well as facile control of adsorption affinity towards water and alcohols especially at low activities relevant to biofuel separation processes.

In an embodiment, a method for forming a hybrid zeolitic imidazolate framework (ZIF) comprises formation steps comprising: preparing a first solution comprising a first imidazolate and a second imidazolate, preparing a second solution comprising a metal ion, and combining the first solution and the second solution to form the hybrid ZIF. In an embodiment, the first imidazolate comprises 2-methylimidazolate and the second imidazolate comprises carboxaldehyde-2-imidazolate. In an embodiment, the hybrid ZIF comprises a first fraction of 2-methylimidazolate and a second fraction of carboxaldehyde-2-imidazolate. In an embodiment, the first fraction is from about 5 to about 90 or any value there between, and the second fraction is 100—the first fraction. In an embodiment, the first fraction is from about 55 to about 70 or any value there between. In an embodiment, the first fraction is from about 25 to about 35 or any value there between. In an embodiment, the first fraction is from about 5 to about 10 or any value there between.

In an embodiment, the metal ion comprises a transition metal. For example, the metal ion comprises a first row transition metal such as nickel, iron, zinc, or cobalt. In an embodiment, the metal ion comprises zinc or cobalt. In an embodiment, the metal ion comprises zinc. In an embodiment, the metal ion comprises cobalt.

In an embodiment, the hybrid ZIF can have n-butane/i-butane diffusion selectivity of at least 1.2 times greater than a non-hybrid ZIF-90. In an embodiment, the ZIF can have a n-butane/i-butane diffusion selectivity of at least 5.7 times greater than a non-hybrid ZIF-90. In an embodiment, the ZIF can have a n-butane/i-butane diffusion selectivity of at least 40 times greater than a non-hybrid ZIF-90.

In an embodiment, the hybrid ZIF can have a butanol/water adsorption selectivity of at least 1.2 times greater than a non-hybrid ZIF-8. In an embodiment, the hybrid ZIF can have a butanol/water adsorption selectivity of at least 5 times greater than a non-hybrid ZIF-8. In an embodiment, the hybrid ZIF can have a butanol/water adsorption selectivity of at least 10 times greater than a non-hybrid ZIF-8.

In an embodiment, the method further comprises an activation step to remove impurities from the hybrid ZIF. In an embodiment, the hybrid ZIF may be activated to remove species such as solvent, unreacted metal ions or unreacted imidazolate remaining in the pores of the ZIF. In an embodiment, the activation step comprises any ZIF activating process commonly known in the art, including but not limited to, heat treating and vacuum degassing. In an embodiment, the activation step comprises heat treating from about 100° C. to about 300° C. In an embodiment, the activating step comprises vacuum degassing from about 100° C. to about 300° C.

In an embodiment, the method further comprises a reaction step to functionalize the hybrid ZIF. In an embodiment, the reaction step comprises exposing the hybrid ZIF to a reactive agent. In an embodiment, the reactive agent can be any reagent known in the art that can undergo a chemical reaction with the hybrid ZIF. For example, the reactive agent may comprise functionalities including, but not limited to, alkyl, amino, chloro, bromo, carbonyl, nitro, sulfonate, hydroxy, hydroxo, aldehyde, organometallic functionalities, and the like. In an embodiment, the reactive agent comprises a functionality selected from the group consisting of alkyl, amino, chloro, bromo, carbonyl, nitro, sulfonate, hydroxy, hydroxo, aldehyde, organometallic functionalities and combinations thereof. In an embodiment, the reactive agent comprises an aldehyde. In an embodiment, the reactive agent comprises an amine.

In an embodiment, a metal-organic framework (MOF) comprises a hybrid ZIF comprising a first imidazolate, a second imidazolate, and a metal ion. In an embodiment, the hybrid ZIF comprises a first fraction of 2-methylimidazolate and a second fraction of carboxaldehyde-2-imidazolate. In an embodiment, the first fraction is from about 5 to about 70 or any value there between, and the second fraction is 100—the first fraction. In an embodiment, the first fraction is from about 55 to about 70 and any value there between. In an embodiment, the first fraction is from about 25 to about 35 and any value there between. In an embodiment, the first fraction is from about 5 to about 10 and any value there between.

In an embodiment, a molecular sieve device comprises a metal-organic framework (MOF) comprising a hybrid ZIF comprising a first imidazolate, a second imidazolate, and a metal ion. In an embodiment, the first imidazolate comprises 2-methylimidazolate and the second imidazolate comprises carboxaldehyde-2-imidazolate.

In an embodiment, the hybrid ZIF comprises a first fraction of 2-methylimidazolate and a second fraction of carboxaldehyde-2-imidazolate. In an embodiment, the first fraction is from about 5 to about 90 or any value there between, and the second fraction is 100—the first fraction. In an embodiment, the first fraction is from about 55 to about 70 or any value there between. In an embodiment, the first fraction is from about 25 to about 35 or any value there between. In an embodiment, the first fraction is from about 5 to about 10 or any value there between.

In a hydrocarbon separation embodiment, the device comprises a feed composition of about 2 mol % to about 95 mol % i-butane, about 2 mol % to about 95 mol % n-butane and mixtures thereof. For example, the feed composition may include about 0 mol % to about 2 mol % ethane, about 0 mol % to about 5 mol % n-propane, about 0 mol % to about 5 mol % i-propane, about 0 mol % to about 5 mol % butenes, about 2 mol % to about 95 mol % i-butane, about 2 mol % to about 95 mol % n-butane, about 0 mol % to about 2 mol % pentenes and about 0 mol % to about 15 mol % pentanes and mixtures thereof.

In an alcohol/water separation embodiment, the device comprises a feed composition of about 0 mol % to about 5 mol % of one or more alcohols, about 2 mol % to about 95 mol % water and mixtures thereof. For example, the feed composition may include about 0 mol % to about 5 mol % methanol, about 0 mol % to about 5 mol % ethanol, about 0 mol % to about 5 mol % propanol, about 0 mol % to about 5 mol % butanol, about 2 mol % to about 95 mol % water and mixtures thereof.

In a hydrocarbon or alcohol/water separation embodiment, the device comprises an operating temperature from about 35° C. to about 95° C. or any value there between. In an embodiment, the operating temperature is about 35° C. In an embodiment, the operating temperature is about 70° C. In an embodiment, the operating temperature is about 95° C.

In a hydrocarbon separation embodiment, the device comprises an operating pressure from about 1 bar to about 14 bar or any value there between. In an embodiment, the operating pressure is about 1 bar. In an embodiment, the operating pressure is about 4 bar. In an embodiment, the operating pressure is about 7 bar. In an embodiment, the operating pressure is about 10 bar. In an embodiment, the operating pressure is about 14 bar.

In an alcohol/water separation embodiment, the device comprises an operating pressure from about 1 bar to about 2 bar or any value there between. In an embodiment, the operating temperature is about 1 bar. In an embodiment, the operating temperature is about 1.5 bar. In an embodiment, the operating temperature is about 2 bar.

These and other objects, features and advantages will become apparent as reference is made to the following detailed description, preferred embodiments, and examples, given for the purpose of disclosure, and taken in conjunction with the accompanying drawings and appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a further understanding of the nature and objects of the present invention, reference should be made to the following detailed disclosure, taken in conjunction with the accompanying drawings, in which like parts are given like reference numerals, and wherein:

FIG. 1A illustrates an exemplary molecular sieve device comprising a hybrid ZIF, wherein the hybrid ZIF is in the form of a membrane grown or deposited on a porous polymeric, ceramic or metallic support according to an embodiment of the present invention;

FIG. 1B illustrates an exemplary molecular sieve device comprising a hybrid ZIF, wherein the hybrid ZIF is in the form of a packed bed of the hybrid ZIF crystals according to an embodiment of the present invention;

FIG. 2 illustrates exemplary SEM images of range of crystals synthesized according to an embodiment of the present invention;

FIG. 3 illustrates charts of D (μm) vs. % Mass, showing the crystal size distributions (CSDs) of: (a) 338 nm ZIF-8, (b) 112.0 μm ZIF-8, (c) 3.9 μm ZIF-8₆₃-90₃₇, (d) 80.4 μm ZIF-8₆₃-90₃₇, (e) 3.5 μm ZIF-8₂₈-90₇₂, (f) 87.1 μm ZIF-8₂₈-90₇₂, (g) 10.3 μm ZIF-8₇-90₉₃, (h) 60.6 μm ZIF-8₇-90₉₃, and (i) 55.7 μm ZIF-90 crystals;

FIG. 4 illustrates a chart of 2θ (°) vs. Intensity (AU), showing powder XRD patterns of the materials shown in FIG. 1 and FIG. 2: (a) 338 nm ZIF-8, (b) 112.0 μm ZIF-8, (c) 3.9 μm ZIF-8₆₃-90₃₇, (d) 80.4 μm ZIF-8₆₃-90₃₇, (e) 3.5 μm ZIF-8₂₈-90₇₂, (f) 87.1 μm ZIF-8₂₈-90₇₂, (g) 10.3 μm ZIF-8₇-90₉₃, (h) 60.6 μm ZIF-8₇-90₉₃, and (i) 55.7 μm ZIF-90 crystals;

FIG. 5 illustrates a chart of ZIF-8 fraction in solution vs. ZIF-8 fraction in powder framework, showing Composition analysis curves of ZIF-8ₓ-90₁₀₀₋ₓ hybrid frameworks obtained by solution ¹H-NMR. Composition curves for smaller (<10 mm) and larger (>50 mm) crystals;

FIG. 6A illustrates exemplary micro-Raman spectra for a crystal in each ZIF-8-90 sample, showing (a) Powder FT-Raman spectra of ZIF-8-90 hybrid framework materials, and (b) micro-Raman spectra from individual ZIF-8-90 crystal;

FIG. 6B illustrates exemplary micro-Raman spectra for a crystal in each ZIF-8-90 sample, showing micro-Raman spectra from individual ZIF-8-90 crystal;

FIG. 7 illustrates a chart of X=100×I_{ZIF8}/(I_{ZIF8}+I_{ZIF90}) vs. ZIF-8 fraction in powder framework, showing composition analysis of ZIF-8-90 hybrid crystals by FT-Raman and micro-Raman;

FIG. 8A illustrates a chart of Pressure (bar) vs. C (mmol/g), showing adsorption isotherms at 35° C. of n-butane for materials with ZIF-8 linker percentage x=100 (pure ZIF-8), 63, 28, 7, and 0 (pure ZIF-90);

FIG. 8B illustrates a chart of Pressure (bar) vs. C (mmol/g), showing adsorption isotherms at 35° C. of i-butane for materials with ZIF-8 linker percentage x=100 (pure ZIF-8), 63, 28, 7, and 0 (pure ZIF-90);

FIG. 9 illustrates a chart of P/P₀ vs. Water Uptake (mmol/g), showing water adsorption isotherms in ZIF. 8-90 hybrid crystals at 308° K, and the water adsorption isotherm of a 50-50 (by mass) physical mixture of ZIF-8 and ZIF-90 crystals at 308° K;

FIG. 10A illustrates a chart of P/P₀ vs. Ethanol Uptake (mmol/g), showing ethanol adsorption isotherms in ZIF.-8-90 hybrid crystals at 308° K;

FIG. 10B illustrates a chart of P/P₀ vs. Butanol Uptake (mmol/g), showing n-butanol adsorption isotherms in ZIF.-8-90 hybrid crystals at 308° K;

FIG. 11 illustrates a chart of Ethanol Uptake (mmol/g) vs. Isosteric Heat of Adsorption (kJ/mol), showing isoteric heat (ΔHᵢₛₒ) of ethanol adsorption for ZIF-8, ZIF-90 and ZIF-8₅₀-90₅₀ hybrid materials;

FIG. 12 illustrates a chart of P/P₀ vs. Ethanol Uptake (mmol/g) and Water Uptake (mmol/g), showing vapor-phase single component adsorption isotherms for water (blue) and ethanol (black) in ZIF-8 (squares) and ZIF-90 (circles) at 323° K;

FIG. 13 illustrates a chart of ZIF-8 linker fraction (x) vs. Diffusivities of n-butane and i-butane (cm2/s), showing Fickian (open symbols) and corrected Maxwell-Stefan (closed symbols) diffusivities of n-butane and i-butane (left axis), and the corresponding n-butane/i-butane diffusion selectivities (right axis) of ZIF-8-90 materials with varying values of x measured at 308° K;

FIG. 14 illustrates a chart of Gradient Strength (G/cm) vs. Signal attenuation (I/I₀), showing PFG NMR signal attenuation curve for water in ZIF-856-9044 at 313° K including single and double exponential data fits; and

FIG. 15 illustrates a chart of ZIF-8 linker fraction (x) vs. Diffusivity (m²/s), showing self-diffusivities and M-S diffusivities of water, methanol, ethanol and n-butanol in ZIF-8 material.

### DETAILED DESCRIPTION OF EMBODIMENTS OF THE INVENTION

The following detailed description of various embodiments of the present invention references the accompanying drawings, which illustrate specific embodiments in which the invention can be practiced. While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the present invention, including all features which would be treated as equivalents thereof by those skilled in the art to which the invention pertains. Therefore, the scope of the present invention is defined only by the appended claims, along with the full scope of equivalents to which such claims are entitled.

One particular advantage of the present invention is the ability to manufacture continuously tunable framework functionality or microporosity. A further advantage is the ability to produce homogenous crystal structure frameworks that would normally form varying crystal structures. The present invention can improve upon the non-hybrid ZIFs. For example, characterization by X-ray diffraction and nitrogen physisorption demonstrates the formation of a set of crystalline ZIF structures that can exhibit adsorption properties different from their parent frameworks. Additionally, continuous control over composition can be possible, as shown by ¹H NMR spectroscopy. Furthermore, the present disclosure relates to a method that can be a facile route whereby chemically and thermally robust ZIFs can be subjected to continuous and tunable alterations in chemical functionality or microporosity by in situ incorporation of various linkers, including various imidazoles and derivatives thereto.

By the method disclosed herein, surface functionalities in ZIF materials can be better controlled and improvements in gas separations can be achieved without severely altering the pores of the material. Additionally, by the methods of the present invention, in situ linker substitution can be performed in various MOFs, including ZIFs, with two different linkers to introduce two different functionalities in the material without changing the crystal structure.

In an embodiment, a method for forming a hybrid zeolitic imidazolate framework (ZIF) comprises formation steps comprising: preparing a first solution comprising a first imidazolate and a second imidazolate, preparing a second solution comprising a metal ion, and combining the first solution and the second solution to form the hybrid ZIF. In an embodiment, the first imidazolate comprises 2-methylimidazolate and the second imidazolate comprise carboxaldehyde-2-imidazolate. In an embodiment, the hybrid ZIF comprises a first fraction of 2-methylimidazolate and a second fraction of carboxaldehyde-2-imidazolate. In an embodiment, the first fraction is from about 5 to about 90 or any value there between, and the second fraction is 100—the first fraction. In an embodiment, the first fraction is from about 55 to about 70 or any value there between. In an embodiment, the first fraction is from about 25 to about 35 or any value there between. In an embodiment, the first fraction is from about 5 to about 10 or any value there between.

In an embodiment, the metal ion comprises a transition metal. For example, the metal ion comprises a first row transition metal such as nickel, iron, zinc, or cobalt. In an embodiment, the metal ion comprises zinc or cobalt. In an embodiment, the metal ion comprises zinc. In an embodiment, the metal ion comprises cobalt.

The hybrid ZIF of the present invention can have an increased adsorption or diffusion selectivity for many molecular pairs as compared to a non-hybrid ZIF. Without being bound by theory, it is thought that better selectivity can be derived from either a change in pores of the hybrid ZIF materials, or a change in surface properties by introducing organic functional groups into the framework. Having a smaller pore can result in better diffusion selectivity for small gas or molecular pairs while changing the organic functional groups can increase the adsorption selectivity. By way of non-limiting examples, the hybrid ZIF can have a greater adsorption or diffusion selectivity for the following molecular pairs: n-pentane/i-pentane, n-butane/i-butane, n-propane/i-propane, butanol/water, propanol/water, ethanol/water, methanol/water and the like.

In an embodiment, the hybrid ZIF can have n-butane/i-butane diffusion selectivity of at least 1.2 times greater than a non-hybrid ZIF-90. In an embodiment, the ZIF can have a n-butane/i-butane diffusion selectivity of at least 5.7 times greater than a non-hybrid ZIF-90. In an embodiment, the ZIF can have a n-butane/i-butane diffusion selectivity of at least 40 times greater than a non-hybrid ZIF-90.

In an embodiment, the hybrid ZIF can have a butanol/water adsoption selectivity of at least 1.2 times greater than a non-hybrid ZIF-8. In an embodiment, the hybrid ZIF can have a butanol/water adsorption selectivity of at least 5 times greater than a non-hybrid ZIF-8. In an embodiment, the hybrid ZIF can have a butanol/water adsorption selectivity of at least 10 times greater than a non-hybrid ZIF-8.

In an embodiment, the method further comprises an activation step to remove impurities from the hybrid ZIF. In an embodiment, the hybrid ZIF may be activated to remove species such as solvent, unreacted metal ions or unreacted imidazolate remaining in the pores of the ZIF. In an embodiment, the activation step comprises any ZIF activating process commonly known in the art, including but not limited to, heat treating and vacuum degassing. In an embodiment, the activation step comprises heat treating from about 100° C. to about 300° C. In an embodiment, the activating step comprises vacuum degassing from about 100° C. to about 300° C.

In an embodiment, the method further comprises a reaction step to functionalize the hybrid ZIF. In an embodiment, the reaction step comprises exposing the hybrid ZIF to a reactive agent. In an embodiment, the reactive agent can be any reagent known in the art that can undergo a chemical reaction with the hybrid ZIF. For example, the reactive agent may comprise functionalities including, but not limited to, alkyl, amino, chloro, bromo, carbonyl, nitro, sulfonate, hydroxy, hydroxo, aldehyde, organometallic functionalities, and the like. In an embodiment, the reactive agent comprises a functionality selected from the group consisting of alkyl, amino, chloro, bromo, carbonyl, nitro, sulfonate, hydroxy, hydroxo, aldehyde, organometallic functionalities and combinations thereof. In an embodiment, the reactive agent comprises an aldehyde. In an embodiment, the reactive agent comprises an amine.

In an embodiment, a metal-organic framework (MOF) comprises a hybrid ZIF comprising a first imidazolate, a second imidazolate, and a metal ion. In an embodiment, the hybrid ZIF comprises a first fraction of 2-methylimidazolate and a second fraction of carboxaldehyde-2-imidazolate. In an embodiment, the first fraction is from about 5 to about 70 or any value there between, and the second fraction is 100—the first fraction. In an embodiment, the first fraction is from about 55 to about 70 and any value there between. In an embodiment, the first fraction is from about 25 to about 35 and any value there between. In an embodiment, the first fraction is from about 5 to about 10 and any value there between.

In an embodiment, the metal ion comprises a transition metal. For example, the metal ion comprises a first row transition metal such as nickel, iron, zinc, or cobalt. In an embodiment, the metal ion comprises zinc or cobalt. In an embodiment, the metal ion comprises zinc. In an embodiment, the metal ion comprises cobalt.

In an embodiment, the hybrid ZIF can have n-butane/i-butane diffusion selectivity of at least 1.2 times greater than a non-hybrid ZIF-90. In an embodiment, the ZIF can have a n-butane/i-butane diffusion selectivity of at least 5.7 times greater than a non-hybrid ZIF-90. In an embodiment, the ZIF can have a n-butane/i-butane diffusion selectivity of at least 40 times greater than a non-hybrid ZIF-90.

In an embodiment, the hybrid ZIF can have a butanol/water adsorption selectivity of at least 1.2 times greater than a non-hybrid ZIF-8. In an embodiment, the hybrid ZIF can have a butanol/water adsorption selectivity of at least 5 times greater than a non-hybrid ZIF-8. In an embodiment, the hybrid ZIF can have a butanol/water adsorption selectivity of at least 10 times greater than a non-hybrid ZIF-8.

In an embodiment, the hybrid ZIF may be purified by an activation step. In an embodiment, the hybrid ZIF may be activated to remove species such as solvent, unreacted metal ions or unreacted imidazolate remaining in the pores of the ZIF. In an embodiment, the activation step comprises any ZIF activating process commonly known in the art, including but not limited to, heat treating and vacuum degassing. In an embodiment, the activation step comprises heat treating from about 100° C. to about 300° C. In an embodiment, the activating step comprises vacuum degassing from about 100° C. to about 300° C.

In an embodiment, the hybrid ZIF may be functionalized by a reaction step. In an embodiment, the reaction step comprises exposing the hybrid ZIF to a reactive agent. In an embodiment, the reactive agent can be any reagent known in the art that can undergo a chemical reaction with the hybrid ZIF. For example, the reactive agent may comprise functionalities including, but not limited to, alkyl, amino, chloro, bromo, carbonyl, nitro, sulfonate, hydroxy, hydroxo, aldehyde, organometallic functionalities, and the like. In an embodiment, the reactive agent comprises a functionality selected from the group consisting of alkyl, amino, chloro, bromo, carbonyl, nitro, sulfonate, hydroxy, hydroxo, aldehyde, organometallic functionalities and combinations thereof. In an embodiment, the reactive agent comprises an aldehyde. In an embodiment, the reactive agent comprises an amine.

In an embodiment, a molecular sieve device comprises a metal-organic framework (MOF) comprising a hybrid ZIF comprising a first imidazolate, a second imidazolate, and a metal ion. In an embodiment, the first imidazolate comprises 2-methylimidazolate and the second imidazolate comprises carboxaldehyde-2-imidazolate. In an embodiment, the hybrid ZIF comprises a first fraction of 2-methylimidazolate and a second fraction of carboxaldehyde-2-imidazolate. In an embodiment, the first fraction is from about 5 to about 90 or any value there between, and the second fraction is 100—the first fraction. In an embodiment, the first fraction is from about 55 to about 70 or any value there between. In an embodiment, the first fraction is from about 25 to about 35 or any value there between. In an embodiment, the first fraction is from about 5 to about 10 or any value there between.

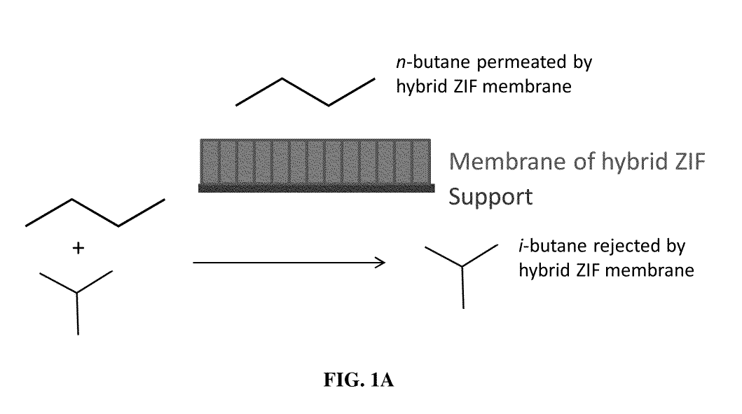

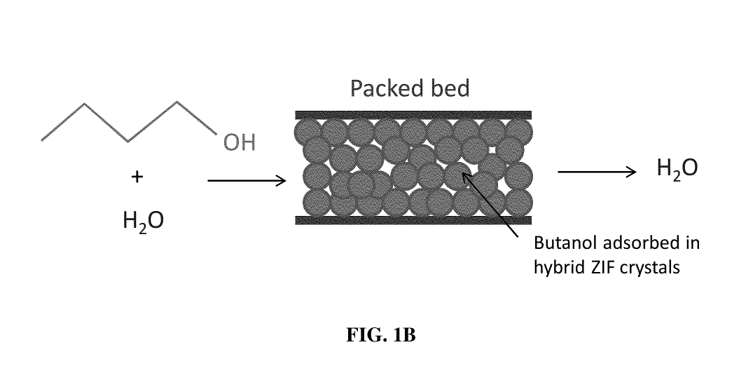

In an embodiment, a molecular sieve separation device comprises a hybrid ZIF, wherein the hybrid ZIF is in the form of a membrane of the hybrid ZIF material grown or deposited on a porous polymeric, ceramic or metallic support. See e.g., FIG. 1A. In FIG. 1A, a hydrocarbon feed composition (e,g., n-butane/i-butane) is contacted with the membrane to separate one or more hydrocarbons (e.g., n-butane). When contacted with a hydrocarbon feed composition (e.g., n-butane/i-butane), the membrane selectively permeates a higher diffusivity component (e.g., n-butane). As shown in FIG. 1A, the membrane rejects a lower diffusivity component (e.g., i-butane).

In an embodiment, a molecular sieve separation device comprises a hybrid ZIF, wherein the hybrid ZIF is in the form of a packed bed of the hybrid ZIF crystals. See e.g., FIG. 1B. In FIG. 1B, an alcohol/water feed composition (e.g., butanol/water) is contacted with the packed bed to separate the alcohol (e.g., butanol) from the water. When contacted with an alcohol/water feed composition (e.g., butanol/water), the packed bed preferentially adsorbs the alcohol (e.g., butanol) over water. As shown in FIG. 1B, the packed bed preferentially permits the water to pass.

In an embodiment, the metal ion comprises a transition metal. For example, the metal ion comprises a first row transition metal such as nickel, iron, zinc, or cobalt. In an embodiment, the metal ion comprises zinc or cobalt. In an embodiment, the metal ion comprises zinc. In an embodiment, the metal ion comprises cobalt.

In an embodiment, the hybrid ZIF can have n-butane/i-butane diffusion selectivity of at least 1.2 times greater than a non-hybrid ZIF-90. In an embodiment, the ZIF can have a n-butane/i-butane diffusion selectivity of at least 5.7 times greater than a non-hybrid ZIF-90. In an embodiment, the ZIF can have a n-butane/i-butane diffusion selectivity of at least 40 times greater than a non-hybrid ZIF-90.

In an embodiment, the hybrid ZIF can have a butanol/water adsorption selectivity of at least 1.2 times greater than a non-hybrid ZIF-8. In an embodiment, the hybrid ZIF can have a butanol/water adsorption selectivity of at least 5 times greater than a non-hybrid ZIF-8. In an embodiment, the hybrid ZIF can have a butanol/water adsorption selectivity of at least 10 times greater than a non-hybrid ZIF-8.

In an embodiment, the hybrid ZIF may be purified by an activation step. In an embodiment, the hybrid ZIF may be activated to remove species such as solvent, unreacted metal ions or unreacted imidazolate remaining in the pores of the ZIF. In an embodiment, the activation step comprises any ZIF activating process commonly known in the art, including but not limited to, heat treating and vacuum degassing. In an embodiment, the activation step comprises heat treating from about 100° C. to about 300° C. In an embodiment, the activating step comprises vacuum degassing from about 100° C. to about 300° C.

In an embodiment, the hybrid ZIF may be functionalized by a reaction step. In an embodiment, the reaction step comprises exposing the hybrid ZIF to a reactive agent. In an embodiment, the reactive agent can be any reagent known in the art that can undergo a chemical reaction with the hybrid ZIF. For example, the reactive agent may comprise functionalities including, but not limited to, alkyl, amino, chloro, bromo, carbonyl, nitro, sulfonate, hydroxy, hydroxo, aldehyde, organometallic functionalities, and the like. In an embodiment, the reactive agent comprises a functionality selected from the group consisting of alkyl, amino, chloro, bromo, carbonyl, nitro, sulfonate, hydroxy, hydroxo, aldehyde, organometallic functionalities and combinations thereof. In an embodiment, the reactive agent comprises an aldehyde. In an embodiment, the reactive agent comprises an amine.

In a hydrocarbon separation embodiment, the device comprises a feed composition of about 2 mol % to about 95 mol % i-butane, about 2 mol % to about 95 mol % n-butane and mixtures thereof. For example, the feed composition may include about 0 mol % to about 2 mol % ethane, about 0 mol % to about 5 mol % n-propane, about 0 mol % to about 5 mol % i-propane, about 0 mol % to about 5 mol % butenes, about 2 mol % to about 95 mol % i-butane, about 2 mol % to about 95 mol % n-butane, about 0 mol % to about 2 mol % pentenes and about 0 mol % to about 15 mol % pentanes and mixtures thereof.

In an alcohol/water separation embodiment, the device comprises a feed composition of about 0 mol % to about 5 mol % of one or more alcohols, about 2 mol % to about 95 mol % water and mixtures thereof. For example, the feed composition may include about 0 mol % to about 5 mol % methanol, about 0 mol % to about 5 mol % ethanol, about 0 mol % to about 5 mol % propanol, about 0 mol % to about 5 mol % butanol, about 2 mol % to about 95 mol % water and mixtures thereof.

In a hydrocarbon or alcohol/water separation embodiment, the device comprises an operating temperature from about 35° C. to about 95° C. or any value there between. In an embodiment, the operating temperature is about 35° C. In an embodiment, the operating temperature is about 70° C. In an embodiment, the operating temperature is about 95° C.

In a hydrocarbon separation embodiment, the device comprises an operating pressure from about 1 bar to about 14 bar or any value there between. In an embodiment, the operating pressure is about 1 bar. In an embodiment, the operating pressure is about 4 bar. In an embodiment, the operating pressure is about 7 bar. In an embodiment, the operating pressure is about 10 bar. In an embodiment, the operating pressure is about 14 bar.

In an alcohol/water separation embodiment, the device comprises an operating pressure from about 1 bar to about 2 bar or any value there between. In an embodiment, the operating temperature is about 1 bar. In an embodiment, the operating temperature is about 1.5 bar. In an embodiment, the operating temperature is about 2 bar.

### EXAMPLES

Materials. 2-methylimidazole (99%, 2-MeIm), zinc nitrate Zn(NO₃)₂.6H₂O (99%) and sodium formate (99%, NaCO₂H) were obtained from Sigma-Aldrich. Carboxyaldehyde-2-imidazole (99%, OHC-Im), dimethylformamide (DMF), and methanol (MeOH) were obtained from Alfa Aesar. Deionized water (DI-H₂O) was produced with a Thermo Scientific 7128.

Synthesis of ZIF-8-90 Hybrid Materials. Different synthesis procedures were used to produce ZIF crystals of different size ranges suitable for hydrocarbon diffusion measurements, as described below:

Synthesis of about 100 μm ZIF-8-90 mixed-linker crystals. Large (about 100 μm average size) ZIF-8-90 mixed-linker crystals were synthesized by modifying the procedure reported by Cravillon et al.¹⁸ A solution of about 0.544 g (8 mmol) of NaCOOH, about x mmol of 2-MeIm (ZIF-8 linker), and about (8-x) mmol of OHC-Im (ZIF-90 linker) was dissolved in about 40 ml of MeOH. The value x was varied from about 0 to about 8. A mixture consisting of about 0.595 g (2 mmol) of Zn(NO₃)₂.6H₂O dissolved in about 40 ml MeOH was poured into 2-MeIm/OHC-Im solution. The resulting solution was heated at about 90° C. for about 24 hours in a sealed glass jar. The large crystals formed on the wall of the jar were collected and washed several times with DI-H₂O and MeOH, and then dried in an oven at about 80° C.

Synthesis of about 10 μm ZIF-8-90 mixed-linker crystals. Small (about 10 μm average size) ZIF-8-90 mixed-linker crystals were synthesized using the method reported by Thompson et al.¹³ A solution of about x mmol of 2-MeIm (ZIF-8 linker), about (20-x) mmol of OHC-Im (ZIF-90 linker) and about 20 mmol of NaCOOH in about 50 ml of MeOH was prepared. The value x was varied from about 0 to about 20. The solution was stirred and heated at about 50° C. until it became clear, and then cooled down to about room temperature. A solution of about 5 mmol of Zn(NO₃)₂.6H₂O in about 50 ml of DI-H₂O was prepared, poured into the first solution, and the resulting mixture was allowed to stir at about room temperature for about 1 hour. ZIF crystals were collected by centrifugation at about 7500 rpm for about 7 minutes, washed in MeOH three times, and dried in an oven at about 80° C.

Synthesis of about 383 nm ZIF-8 crystals. ZIF-8 crystals of about 338 nm average size were synthesized using the method reported by Lai et al.⁷ About 22.7 g (276.5 mmol) of 2-MeIm (ZIF-8 linker) was added to about 70 mL DI-H₂O and stirred with a magnetic bar until the solution became clear at about room temperature. A mixture consisting of about 1.17 g (3.7 mmol) of Zn(NO₃)₂.6H₂O dissolved in about 18 mL DI-H₂O was poured into the 2-MeIm/DI-water solution and stirred at about room temperature for about 12 hours. The resulting milky solution was centrifuged at about 9000 rpm for about 15 minutes followed by washing with MeOH and DI-H₂O three times to collect the ZIF crystals, which were then dried in an oven at about 80° C.

Synthesis of about 55.7 μm ZIF-90 crystals. ZIF-90 crystals of about 55.7 μm average size were synthesized by modifying the procedure reported by Gee et al.¹⁴ About 3.84 g (40 mmol) of OHC-Im (ZIF-90 linker) and about 2.97 g (10 mmol) of Zn(NO₃)₂.6H₂O were added to about 100mL DMF. The solution was heated to about 120° C. while stirring for about 10 minutes in a glass jar. The light-orange colored solution was poured into a wide-necked bottle and capped for about 24 hours at about room temperature. The large crystals on the wall of the jar were collected, washed with DI-H₂O and MeOH three times, and dried in an oven at about 80° C.

Characterization Methods. XRD patterns were measured on a PANalytical X′Pert Pro diffractometer at about room temperature using Cu Ka radiation of λ=0.154 nm and a scanning range of 5-40° N. Crystal size distribution (CSD) analysis was conducted with a Protein Solutions DynaPro DLS instrument, a Hitachi SU 8010 SEM, and a Nikon Eclipse 50i optical microscope. The CSD of about 338 nm ZIF-8 was obtained by DLS. The ZIF-8 powder was dispersed by sonication in a filtered MeOH solution for about 5 minutes. The colloidal suspension was inserted into a cuvette via a 5 μm syringe filter for DLS measurements. CSDs of about 1-10 um ZIF-8-90 materials were measured from multiple SEM images to obtain sample sizes of more than about 200 crystals in each case. CSD of ZIF crystals greater than about 30 um in size were obtained by optical microscopy. The samples were dispersed on a slide glass and the CSD was measured from about 200 crystals in each case. Since large ZIF crystals are highly faceted, the equivalent spherical crystal radius was taken to be that of the smallest circle that encompasses the entire crystal.

Solution ¹H-NMR measurements were performed with a Bruker 400 MHZ spectrometer after digesting the ZIF crystals in D4-acetic acid (CD₃COOD). To determine the fraction of each imidazole linker in the ZIF materials, the integrated peak area of the methyl protons of 2-MeIm (chemical shift 2.65 μm) was normalized to that of the aldehyde proton of OHC-Im (9.84 μm). The chemical shifts of both imidazole linkers was referenced to the chemical shift (2.30 μm) of D4-acetic acid. Powder FT-Raman spectroscopy was performed with a Bruker Vertex 80v FTIR/RAM II FT-Raman Analyzer in open atmosphere and a He/Ne red laser (1054 nm). Raman microscopy of individual ZIF crystals was carried out using a Horiba Jobin-Yvon HR-800 dispersive spectrometer with an 1800 1/mm grating and a green laser (532nm). A spot size of 2.5 μm was used. Numerical integration of FT-Raman and micro-Raman peak areas was carried out with the instrument software. The 2-MeIm and OHC-Im peaks were background-subtracted using a polynomial, and then fitted with mixed Gaussian—Lorentzian functions to obtain the integrated peak areas.

Adsorption and Diffusion Measurements. For Pulsed field gradient (PFG)-NMR measurements, samples were prepared in standard 5mm o.d. NMR tubes. Sample loadings were calculated from adsorption isotherms given by Zhang et al.¹⁵ Loadings were limited at about 10-15% below saturation loading for all adsorbates. This range was chosen to avoid bulk condensation of liquid adsorbates in the NMR tube. The sample tubes were capped, thoroughly sealed using Parafilm and allowed to equilibrate for about 48 hours before experiments were performed.

The diffusivity experiments were performed using a Bruker Advance III NMR spectrometer equipped with a diff-50 diffusion accessory operating at a ¹H frequency of 400 MHz. The stimulated spin echo pulse sequence was used to collect the NMR data and processed using Bruker's TopSpin™ software package. It was verified that the experimental conditions for the Diffusion NMR experiment were chosen such that the experiment measures the intramolecular diffusion (namely, the average displacement of molecules during the diffusion time δ is significantly smaller than the crystallite size).

Adsorption isotherms for water and alcohols were collected using a VTI SA Vapor Sorption Analyzer (TA Instruments). Approximately 10-20 mg of the samples were used for each experiment. The samples were degassed in situ at about 105° C. for up to about 8 hours in an ultrapure N₂ stream. The relative vapor pressure of each adsorbate was varied between the limits of about 0.04 and about 0.9 in discrete steps. Equilibrium was assumed to be achieved if less than about 0.003% weight change was observed in about 5-minute intervals.

The n-butane and i-butane transport diffusivities and adsorption isotherms were measured with a volumetric (pressure decay) apparatus. A known amount of ZIF sample was sealed into a 0.5 μm filter element and installed in the sample chamber. The volumes of the sample chamber and reservoir chamber are precisely known. It was previously determined that the inventors' experiments satisfied the criterion for isothermal macroscopic diffusion. The apparatus was placed in a silicone oil bath equipped with a circulator for temperature control. The sample was degassed under vacuum at about 150° C. for about 12 hours and then maintained for about 12 hours at about 35° C. The vacuum was then isolated, and a known quantity of hydrocarbon gas was injected into the reservoir chamber. The valve connecting the sample and reservoir chambers was then opened. Sensitive pressure transducers attached to the sample and reservoir chambers were used to measure the pressure changes over time, occurring due to adsorption. The data were converted to uptake curves using a virial equation of state.

Crystal Size Distributions. To successfully measure intracrystalline diffusivities that vary over several orders of magnitude, control over the mixed-linker ZIF-8-90 crystal size is necessary. For example, the uptake of i-butane is slow enough to allow measure reliable intracrystalline diffusivity measurements at about 35° C. with about 1-10 μm crystals, whereas crystals larger than about 50 μm are required to accurately measure n-butane diffusivities. ZIF-8-90 mixed-linker crystals of diameters ranging from about 338 nm to about 120 μm were synthesized for uptake measurements. The mixed-linker crystals were synthesized solvothermally, and equimolar amounts of sodium formate (NaCOOH) and organic linkers, such as 2-methylimidazole (2-MeIm) and carboxyaldehyde-2-imidazole (OHC-Im), were used to obtain a macroscopically random linker distribution in the framework. Thermodynamically, the Zn²⁺ metal center favors crystallization with 2-MeIm than with OHC-Im.¹⁹ However, in the presence of sufficient concentrations of sodium formate (NaCOOH), both linkers will be largely deprotonated before addition of Zn²⁺ ions.¹⁸ This allows kinetic control of the metal-linker coordination reaction, and allows the formation of mixed-linker frameworks of continuously variable compositions.

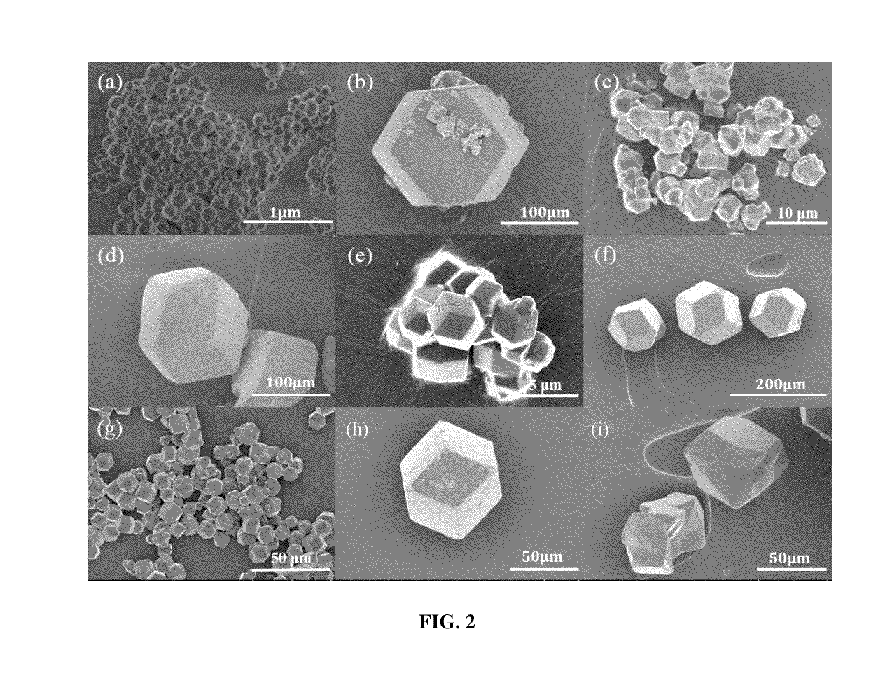

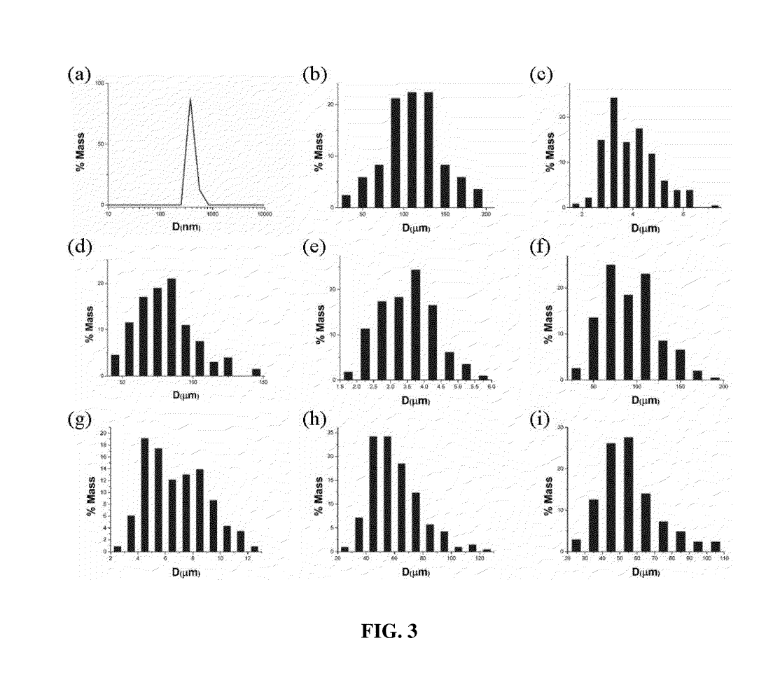

FIG. 2 shows example SEM images of the range of crystals synthesized, and FIG. 3 shows the crystal size distributions obtained from DLS, SEM, or optical microscopy. In FIG. 3, the crystal sizes are number averages obtained from the CSDs, and examplary SEM images of these crystals are shown in FIG. 2. The CSDs were obtained by DLS for sample (a), by SEM for samples (c, e, g), and by optical microscopy for samples (b, d, f, h, i).

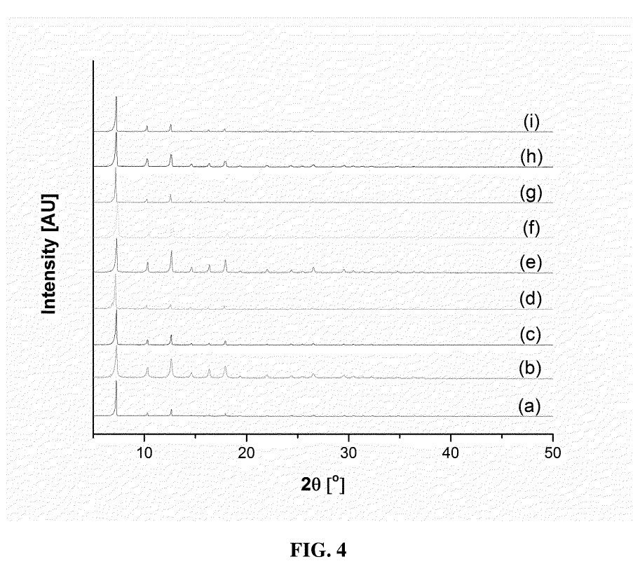

The ZIF-8/ZIF-90 structure topology of all the materials was confirmed by powder XRD, as shown in FIG. 4. In FIG. 4, the crystal sizes are number averages obtained from the CSDs as shown in FIG. 3.

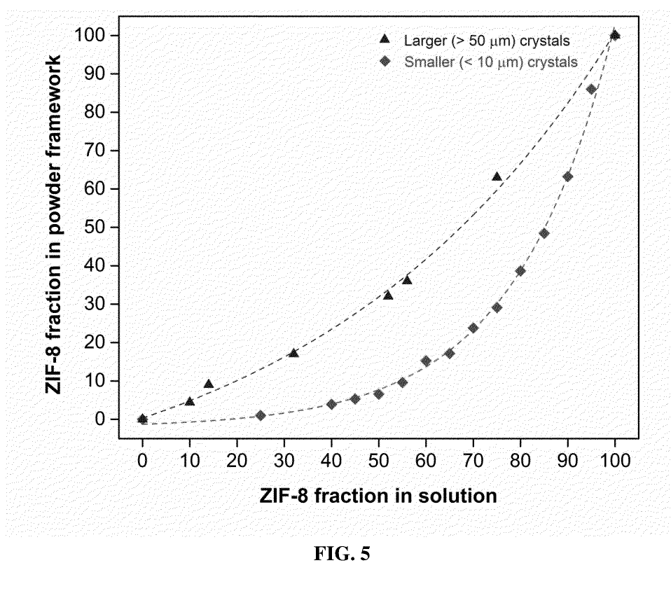

Composition. In general, one expects thermodynamic and kinetic differences in the incorporation of the two different linkers in the ZIF crystal structure. As a result, the fraction (x) of ZIF-8 linkers in the crystallized material is not identical to that originally present in the synthesis solution. It is therefore necessary to establish the “composition curve” that relates the two quantities and allows selection of the appropriate synthesis solution for a particular hybrid ZIF-8-90 material. Solution-phase ¹H-NMR spectroscopy is a reliable tool for this purpose, and the composition curves thus determined are shown in FIG. 5. Due to the different synthesis conditions (and hence different crystallization characteristics) used in the synthesis of “smaller” (<about 10 μm) and “larger” (>about 50 μm) ZIF-8-90 crystals, the composition curves are different for the two cases. Overall, it is seen that 2-MeIm is incorporated into the frameworks at lower fractions than present in the initial reactant solution. The detailed data shown in FIG. 5 is also in good agreement with initial data for small crystals of ZIF-8-90 materials.¹³ Based upon the foregoing results, it is clear that ZIF-8-90 hybrids of any composition and a large range of average crystal sizes can be synthesized by the combination of techniques used in this work.

The XRD patterns of ZIF-8-90 materials are all essentially identical, as shown in FIG. 4, because all the materials have the same framework topology and only a small difference in electron density. In these circumstances, it is difficult to obtain direct evidence of compositional variations by powder diffraction techniques. In a previous work,¹³ indirect evidence (via N₂ physisorption measurements) was provided, suggesting that the crystallized ZIF-8-90 materials may be true hybrids and not physical mixtures of ZIF-8 and ZIF-90 crystals. However, conclusive evidence of hybrid crystal formation, as well as the distribution of the ZIF-8 linker fraction (x) across individual crystals, can only be obtained from a microanalytical technique. Here, the inventors used a comparative approach based upon micro-Raman and powder FT-Raman spectroscopy.

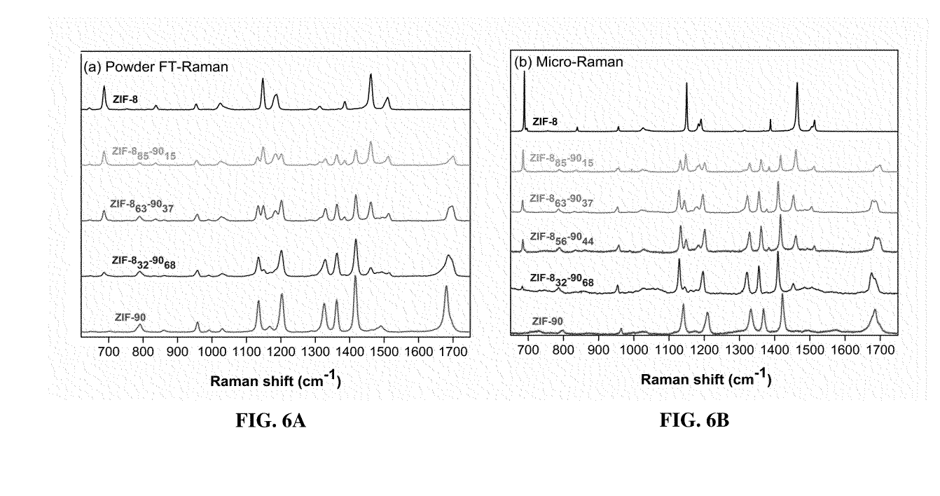

FIGS. 6A-6B show powder FT-Raman spectra and exemplary micro-Raman spectra for several ZIF-8-90 materials. FIG. 6A shows powder FT-Raman spectra from several ZIF-8-90 materials. The peaks at 680 cm⁻¹ (ring puckering of 2-MeIM) and 1680 cm⁻¹(C═O stretching vibration of OHC-Im) were used as signatures⁷⁻⁹ of the ZIF-8 and ZIF-90 linkers, respectively. Then, the integrated areas (I_{zIF8} and _{Iz1F90}) of these peaks in each spectrum were obtained, and the normalized quantity X=100×I_{ZIF8}/(I_{ZIF8}+I_{ZIF90}) were used as a measure of the percentage of ZIF-8 linker in the framework. A similar procedure may be carried out using micro-Raman spectra collected from at least six randomly selected individual crystals in each sample, and at three different locations on each selected crystal.

FIG. 6B shows exemplary micro-Raman spectra from a crystal in each ZIF-8-90 sample. The quantity X allows the cancellation of sample size effects, but is not the exact equivalent of the ZIF-8 linker fraction (x) because of the different polarizabilities of the two characteristic linker vibrations. However, if the crystals are true hybrids, the value of X obtained macroscopically from a powder FT-Raman measurement should be similar to that obtained microscopically from micro-Raman measurements from individual crystals and locations in the sample. Moreover, a small standard deviation of X (as obtained from averaging the micro-Raman measurements over multiple crystals) would denote a highly uniform value of the ZIF-8 linker percentage x across crystals in a given powder sample.

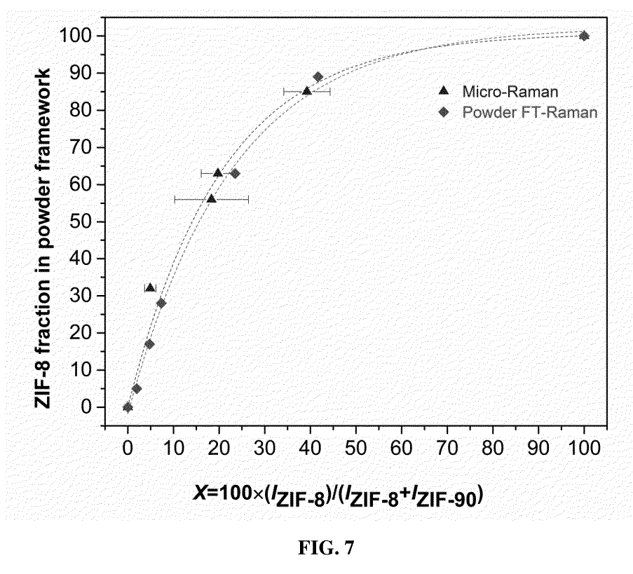

FIG. 7 plots the values of X obtained from FT-Raman and micro-Raman measurements versus the values of x obtained previously from ¹H-NMR measurements. In FIG. 7, the quantity X=100×I_{ZIF8}(I_{ZIF8}+I_{ZIF90}) is obtained from the Raman measurements whereas the quantity y is the corresponding ZIF-8 linker fraction obtained from ¹H-NMR. The error bars shown for the micro-Raman curve represent the standard deviation in X across at least six different crystals of the sample and three different locations in each crystal. The FT-Raman and micro-Raman techniques are in close agreement, thereby providing clear evidence that the crystals are true mixed-linker hybrids. The generally small standard deviations (represented as horizontal error bars in FIG. 7) of X also indicate good compositional uniformity of the ZIF-8-90 crystals. It is important to note that the above discussion does not provide insight on the molecular-level distribution of the two different linkers within the ZIF crystals. As recently shown for a different metal-organic framework (MOF) system, such understanding can be obtained through a combination of NMR spectroscopy and structure modeling.²⁰ The present work, on the other hand, is focused on clearly demonstrating the role of linker substitution in obtaining large changes in adsorption and diffusion behavior.

Adsorption. Volumetric uptake profiles of n-butane and i-butane were collected at about 308° K for five materials with x=100, 63, 28, 7, and 0, representing decreasing ZIF-8 linker content and increasing effective pore size from pure ZIF-8 to pure ZIF-90.

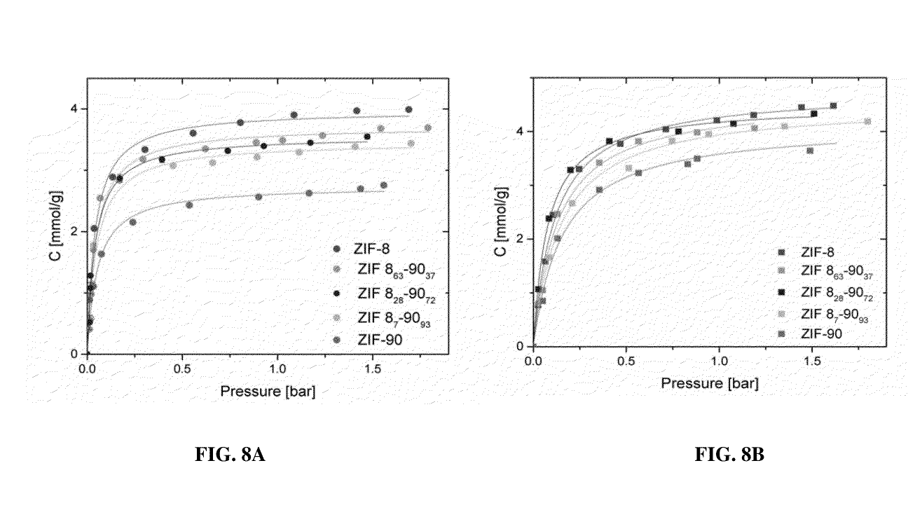

FIGS. 8A-8B show the adsorption isotherms obtained at equilibrated conditions up to a pressure of about 1.8 bar. The data are fitted to Langmuir isotherms. In FIGS. 8A-8B, the solid lines represent Langmuir model fits, with Langmuir model parameters shown in Table 1. The fitted Langmuir capacity (Cₛ), affinity constant (b) and the Henry constant (K =Cₛb) are tabulated in Table 1 below.

TABLE 1
Langmuir model parameters and Henry constants at 35° C.

| | n-butane | | | i-butane | | |
|---|---|---|---|---|---|---|
| | | | K | | | K |
| | Cₛ | b | mmol/ | Cₛ | b | mmol/ |
| Material | mmol/g | 1/bar | (g · bar) | mmol/g | 1/bar | (g · bar) |
| ZIF-8 | 4.0 ± 0.1 | 24 ± 2 | 96 ± 8 | 4.8 ± 0.1 | 9 ± 1 | 41 ± 2 |
| ZIF-8₆₃- | 3.7 ± 0.1 | 22 ± 3 | 82 ± 10 | 4.6 ± 0.1 | 8 ± 1 | 36 ± 5 |
| 90₃₇ | | | | | | |
| ZIF-8₂₈- | 3.6 ± 0.1 | 24 ± 3 | 85 ± 11 | 4.5 ± 0.1 | 13 ± 1 | 59 ± 3 |
| 90₇₂ | | | | | | |
| ZIF-8₇- | 3.5 ± 0.1 | 20 ± 3 | 71 ± 12 | 4.5 ± 0.1 | 7 ± 1 | 30 ± 2 |
| 90₉₃ | | | | | | |
| ZIF-90 | 2.8 ± 0.1 | 19 ± 1 | 52 ± 4 | 4.2 ± 0.1 | 6 ± 1 | 26 ± 3 |

There is a general increase in the Langmuir capacity and Henry constant with the fraction of ZIF-8 linker, due to the more favorable interactions of alkanes with the methyl groups of the 2-MeIm linker. All the ZIF-8-90 materials slightly favor i-butane adsorption over n-butane. Overall, the adsorption properties show moderate changes as a function of x, as expected for adsorption of alkanes in ZIF materials which is governed by van der Waals interactions of the alkyl groups with the framework.

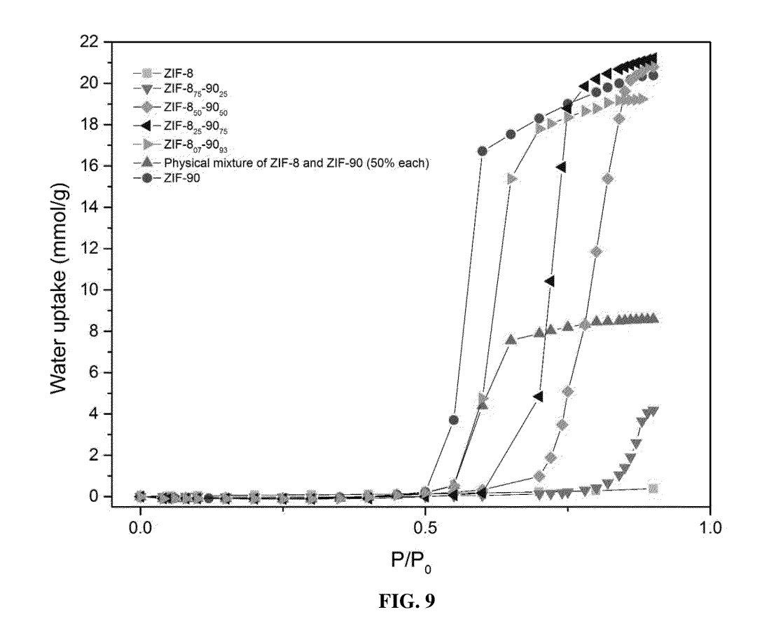

However, drastic changes are seen in the adsorption of water and alcohols upon tuning the ZIF-8-90 composition. FIG. 9 shows water vapor adsorption isotherms in ZIF-8-90 materials at about 308° K. It is clear that the hydrophobicity of ZIF materials can be tuned by controlling the composition of different linkers in the hybrid framework. As the fraction of hydrophilic carbonyl groups in the structure increases from pure ZIF-8 to pure ZIF-90, water uptake starts to occur at lower relative pressures. The water adsorption isotherm for a physical 50-50 wt% mixture of ZIF-8 and ZIF-90 crystals is entirely different from that of a ZIF-8₅₀-90₅₀ hybrid material. This is further corroboration that the 2-MeIm and OHC-Im linkers are forming true hybrid ZIFs.

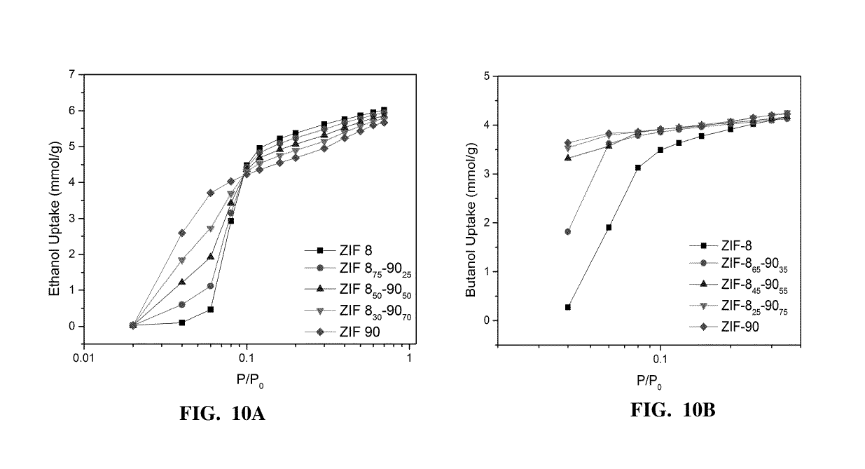

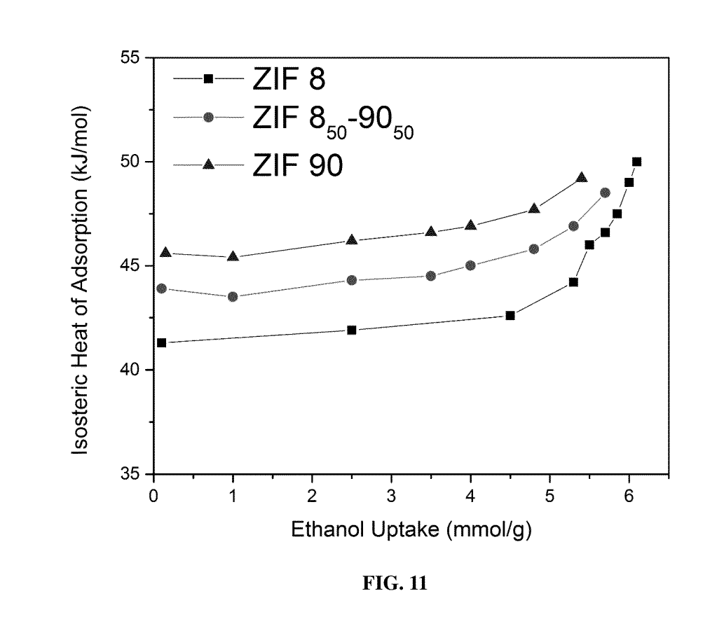

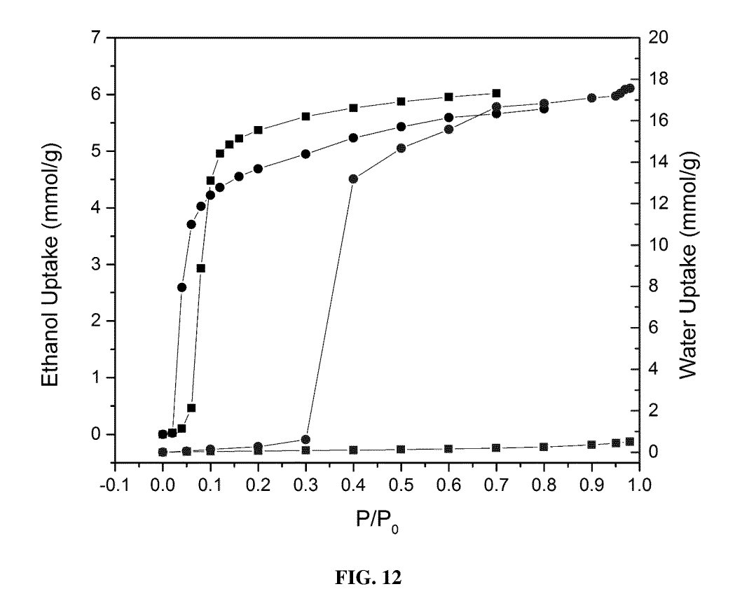

FIGS. 10A-10B show ethanol and n-butanol adsorption isotherms at about 308° K. It is clear that the organophilicity of ZIFs can be tuned significantly, especially in the initial plateau region at low relative pressures, by adjusting the linker fraction (x). This region of low activity is significant for the concentration of alcohols (and other organics) from dilute aqueous solutions, a problem often encountered in biofuel and biobased chemical production. For example, ethanol uptake into pure ZIF-8 only occurs at P/P₀>0.06. By progressively replacing methyl groups with carbonyl groups, the hybrid ZIF-8-90 framework attracts a much larger number of ethanol molecules at low pressures.

The isosteric heat (AH₀) of ethanol adsorption for ZIF-8, ZIF-90 and ZIF 8₅₀-90₅₀ is shown in FIG. 11. ZIF-90 has the highest ΔHᵢₛₒ at infinite dilution due to the polar aldehyde (—CHO) group. Moreover, the ΔHᵢₛₒ values increase slightly during ethanol uptakes up to 5 mmol/g for all three ZIFs. This indicates energetic homogeneity of the hybrid ZIF framework to ethanol adsorption, and relatively weak adsorbate-adsorbate interactions in the low-pressure region. For ethanol uptakes higher than 5 mmol/g, ΔHᵢₛₒ increases more significantly with loading due to the more closely packed and hydrogen-bonded adsorbate molecules confined in the framework. The experimentally derived ΔHᵢₛₒ values for pure ZIF-8 and ZIF-90 are in good agreement with existing molecular simulation data.²¹

Obtaining isosteric heat of adsorption. The isosteric heat of adsorption is derived from the Clausius-Clapeyron equation:

$$Δ {H}_{iso} = {{RT}^{2} ( \frac{\partial ln p}{\partial T} )}_{T} \qquad ( 1 )$$

where ΔHᵢₛₒ is the isosteric heat of adsorption and T indicates constant equilibrium adsorption quantities. The isosteric heat of adsorption ΔHᵢₛₒ can be calculated by measuring adsorption isotherms at different temperatures and employing the thermodynamic relationship of equation (1). A plot of lnP against (1/7) at constant adsorption uptakes yields a straight line with a slope equal to (−ΔHᵢₛₒ/R).

Adsorption (Cont.). The characteristic S-shape isotherms (see FIGS. 8, 9A &11) are not uncommon for ZIF materials with inherent structural flexibility that can undergo structural transformation induced by temperature, pressure, or guest molecules. A “gate-opening” effect, i.e., the reorientation of the imidazolate linkers, was proposed to explain the inflections in experimental N₂ physisorption isotherms at about 77° K.²² The inflection points are at relative pressures of about 5×10⁻³ for ZIF-8 and about 10⁻⁴ for ZIF-90. However, the alcohol adsorption mechanism is different from that of cryogenic nitrogen (N₂) adsorption and is not related to the gate-opening effect. In ZIF-8, the gate-opening effect can only be obtained at high hydrostatic pressures or ultra-low-pressure N₂ adsorption at about 77° K. More importantly, the gate-opening effect is characterized by a hysteresis loop that marks the transformation between a less porous and a more porous phase induced by guest molecules. In the case of ethanol adsorption in ZIF-8, no desorption hysteresis was observed.¹⁶ The S-shape ethanol adsorption isotherm in ZIF-71, a hydrophobic ZIF with zeolite RHO-topology, has been identified as a cluster formation and cage-filling mechanism via molecular simulations. A more recent simulation study revealed that the cluster formation and cage-filling mechanism also holds for the adsorption of normal alcohols (methanol, ethanol, propanol and butanol) in zeolite SOD-topology ZIF materials such as ZIF-8.²¹ At low loadings, the alcohol molecules form clusters at preferential adsorption sites around the organic linkers. With increasing vapor pressure, cage-filling occurs with a large saturation uptake of alcohol molecules. Framework flexibility has a negligible effect on equilibrium alcohol adsorption in ZIF-8 and ZIF-90.¹⁴ These studies indicate that the tunability of water and alcohol adsorption in hybrid ZIF-8-90 frameworks is likely a direct result of the tunable linker fractions, and not an indirect result of changes in framework flexibility or gate-opening.

Diffusion. To clearly isolate the effect of pore tunability on molecular sieving in ZIF-8-90 materials, the inventors focused on the low-pressure regime wherein adsorbate-adsorbate interactions have minimal impact. In the case of the two hydrocarbon isomers n-butane and i-butane, the transport (i.e., Fickian) diffusivities are obtained by fitting the initial linear gravimetric uptake curves with the analytical model for uptake in spherical particles of given CSD, as described below.

Obtaining transport diffusivities from uptake data. Transport diffusivities may be calculated by fitting the initial linear uptake rate with an approximate analytical solution for uptake in spherical particles at constant gas pressure:

$$\frac{{M}_{t}}{{M}_{∞}} = \frac{6}{R} \sqrt{\frac{D}{π}} \cdot \sqrt{t} \qquad ( 2 )$$

In equation (2), Mₜ and M_{∞} are moles adsorbed by sample at time t and time goes to infinity (mmol), respectively. R is equivalent average radius of the spherical sample (cm) and D is transport diffusivity (cm²/s). However, accurate diffusivities may not be obtained using the average crystal size. Further, the uptake process always involves changes in gas pressure with uptake time, which means that equation (2) is not strictly valid.

Thus, a more detailed model taking into account the CSD and the non-constant pressure boundary condition was used to calculate transport diffusivity:

$$\begin{matrix}\frac{{M}_{t}}{{M}_{∞}} \approx {\sum }_{i} {X}_{i} ( 1 + α ) [ 1 - \frac{{γ}_{1}}{{γ}_{1} + {γ}_{2}} e erfc \{ \frac{3 {γ}_{1}}{α} {( \frac{{D}_{t }}{{R}_{i}^{2}} )}^{1 / 2} \} - \frac{{γ}_{2}}{{γ}_{1} + {γ}_{2}} e erfc \{ \frac{3 {γ}_{2}}{α} ( \frac{{D}_{t}}{{R}_{i}^{2}} ) \} ] & ( 3 ) \\ α = \frac{1}{Λ} - 1 & ( 4 ) \\ {γ}_{1} = \frac{1}{2} \{ {( 1 + \frac{4}{3} α )}^{1 / 2} + 1 \} & ( 5 ) \\ {γ}_{2} = {γ}_{1} - 1 & ( 6 ) \\ e erfc ( z ) = exp ( {z}^{2} ) \times erfc ( z ) & ( 7 )\end{matrix}$$

In equations (3) through (7), Mₜ and M_{∞} are moles adsorbed by sample at time t and time goes to infinity (mmol), respectively. R is equivalent average radius of the spherical sample (cm) and Dₜ is transport diffusivity (cm²/s). A is the fraction of adsorbate finally adsorbed by the crystal, and Xᵢ is the mass fraction of the crystals with a radius of Rᵢ.

Diffusion (Cont.). To further elucidate the role of tunable molecular sieving, the inventors obtained the corrected Maxwell-Stefan (M-S) diffusivity from the transport diffusivity. The M-S diffusivity captures the intrinsic rate of hopping of individual molecules through the pore windows of the material.

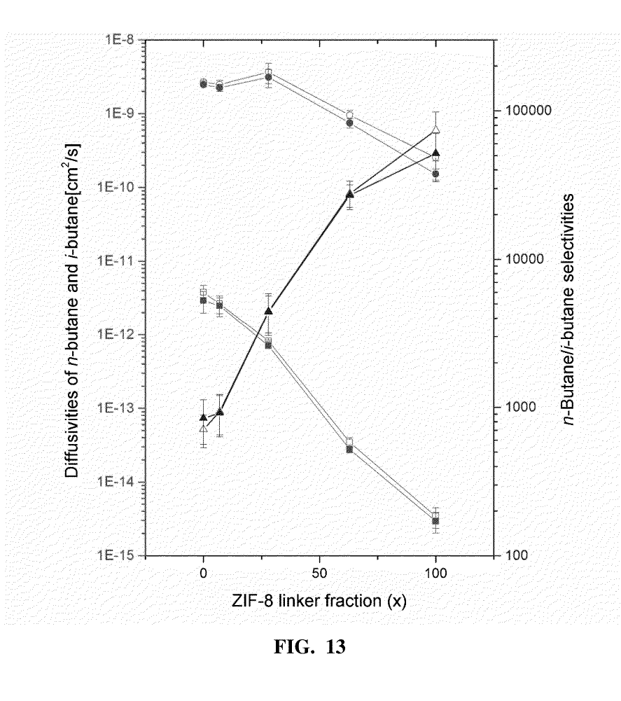

FIG. 13 shows the butane isomer transport diffusivities, M-S diffusivities, and the corresponding n-butane/i-butane diffusion selectivities of ZIF-8-90 materials. The data is provided in Tables 2 and 3 below.

TABLE 2
Transport (Fickian) diffusivities of n-butane and i-butane in
ZIF-8-90 mixed-linker materials at 35° C., measured at
an initial pressure of 0.2 bar and a final pressure of 0.03 bar.

| | n-butane | i-butane | |
|---|---|---|---|
| | Diffusivity | Diffusivity | |
| Material | [10⁻⁹ cm²/s] | [10⁻¹² cm²/s] | Selectivity |
| ZIF-8 | 0.25 ± 0.03 | 0.003 ± 0.002 | 70000 ± 20000 |
| ZIF 8₆₃-90₃₇ | 0.95 ± 0.15 | 0.035 ± 0.005 | 28000 ± 6000 |
| ZIF 8₂₈-90₇₂ | 3.7 ± 1.2 | 0.82 ± 0.02 | 4000 ± 1000 |
| ZIF 8₇-90₉₃ | 2.5 ± 0.3 | 2.6 ± 0.7 | 900 ± 300 |
| ZIF-90 | 2.7 ± 1.2 | 3.8 ± 0.9 | 700 ± 200 |

In Table 2, the error bars are based upon measurements using three independently synthesized powder samples of each ZIF material.

TABLE 3
Corrected Maxwell-Stefan (M-S) diffusivities of n-butane
and i-butane in ZIF 8-90 mixed-linker materials at 35° C.

| | n-butane | i-butane | |
|---|---|---|---|
| | Diffusivity | Diffusivity | |
| Material | [10⁻⁹ cm²/s] | [10⁻¹² cm²/s] | Selectivity |
| ZIF-8 | 0.15 ± 0.03 | 0.003 ± 0.001 | 50000 ± 20000 |
| ZIF- 8₆₃-90₃₇ | 0.7 ± 0.1 | 0.028 ± 0.003 | 27000 ± 5000 |
| ZIF- 8₂₈-90₇₂ | 3.1 ± 0.9 | 0.71 ± 0.06 | 4000 ± 1000 |
| ZIF- 8₇-90₉₃ | 2.3 ± 0.3 | 2.5 ± 0.7 | 900 ± 300 |
| ZIF-90 | 2.5 ± 0.2 | 2.9 ± 1.0 | 800 ± 300 |

The M-S diffusivities in Table 3 were obtained from the transport diffusivities shown in Table 1, as described below.

Fitting of adsorption isotherms. The adsorption isotherms were fitted by the Langmuir model, with parameters listed in Table 1:

$$C ( p ) = \frac{{C}_{s} bp}{1 + bp} \qquad ( 8 )$$

In equation (8), p is the equilibrium pressure of sample chamber (bar), C is the adsorbate concentration in the sample (mmol/g), Cₛ is the Langmuir capacity constant (mmol/g) and b is the Langmuir affinity constant (1/bar).

Obtaining M-S diffusivities from transport diffusivities. The corrected M-S diffusivity was calculated as follows:

$$D = {D}_{0} \frac{d ln p}{d ln C ( p )} \qquad ( 9 )$$

In equation (9), D is the transport diffusivity (cm²/s) and D₀ is the corrected M-S diffusivity (cm²/s). The correction factor on the right-hand side of equation (9) is the derivate of the isotherm and was calculated from the adsorption isotherm data in logarithmic coordinates.

Diffusion (Cont.). From Table 3, it is clear that the n-butane and i-butane transport diffusivities can be tuned continuously over 2-3 orders of magnitude by variation of the ZIF-8 linker fraction (x). The n-butane diffusion selectivity over i-butane can be tuned between about 900 and about 50000. A decreasing value of x leads to an increase in the effective pore size and allows faster hopping of both butane isomers through the pore windows. All the ZIF 8-90 materials have quite a sharp intrinsic selectivity for n-butane (kinetic diameter 0.43 nm) over i-butane (0.5 nm).

However, other important considerations drive the selection of an optimum material for membrane-based separation of butane isomers based upon FIG. 8. Materials close to ZIF-8 have impractically low n-butane diffusivities. Hence, materials closer to ZIF-90 are desired. The n-butane diffusivity appears to reach a plateau when x decreases below 28, whereas such an effect is not observed with i-butane. This is likely due to an uncertainty in determining accurate n-butane diffusivities in the larger-pore materials, due to fast n-butane diffusion and resulting contamination of the data by external and surface mass transfer resistances even in the largest crystals used in this work. On the other hand, the data may also include a real effect, i.e., additional increase in the pore size at smaller values of x can no longer affect significantly the diffusion of the smaller hydrocarbon n-butane, whereas the larger i-butane continues to feel the limiting effect of the pore size. In either case, FIG. 8 shows that materials close to ZIF-90 (0<x<30) are appropriate choices for n-butane/i-butane separation based on molecular sieving, since they combine high n-butane diffusivity (>2×10⁻⁹ cm²/s) and high selectivity (at least about 900 to about 6000, considering the measured n-butane diffusivities as lower-bound values).

In the case of water and alcohols, the self-diffusivities of the three smaller molecules (i.e., water, methanol and ethanol) were measured by PFG-NMR and the larger molecule (i.e., n-butanol) was measured by gravimetric uptake as shown in FIG. 15. The water, methanol, and ethanol self-diffusivities were measured by PFG-NMR at 313° K.

Due to their high diffusivities, gravimetric uptake measurements of diffusion were not feasible in these cases even with the largest crystals available. The PFG-NMR signal attenuation data were fitted to a double-exponential curve to obtain the self-diffusivity coefficient. The dominant fast diffusion component in the decay curve reflects the diffusion of the water/alcohol while the minor component has been attributed to a background signal from remaining solvent.

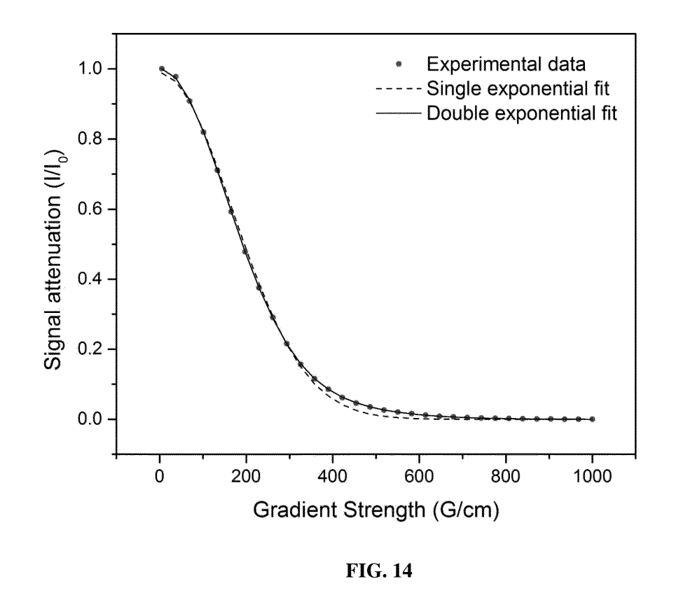

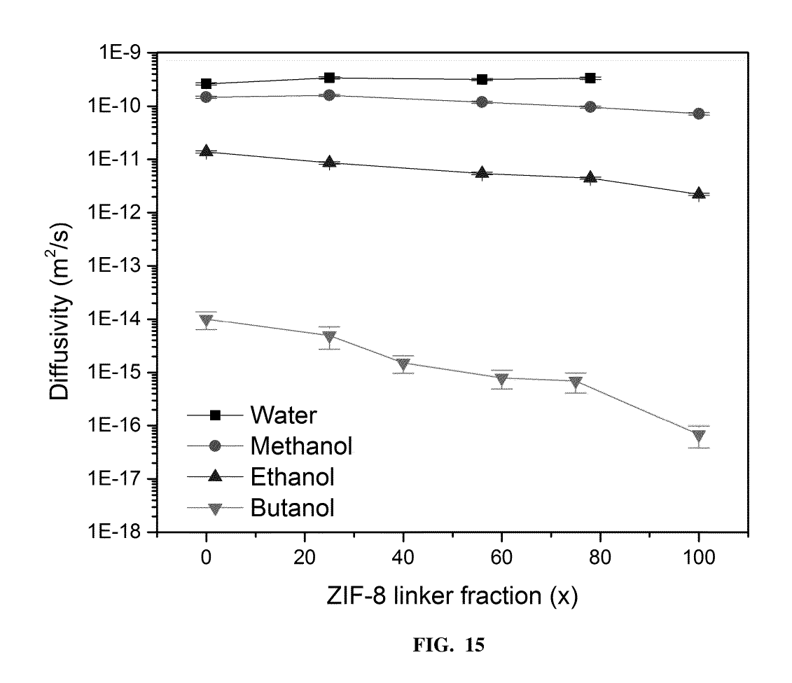

Representative fits are shown in FIG. 14. The diffusivity of water in ZIF-8 was not measured due to its high hydrophobicity. The bulkiest of the four molecules (n-butanol) exhibited a very poor signal-to-noise ratio in PFG-NMR data, which is due to a restricted rotational motion of the molecule, which manifests itself in a short transversal relaxation constant (T₂). Hence the diffusivity was measured gravimetrically. The uptake data was then analyzed with methods similar to those used for the hydrocarbon isomers, as discussed below.

Obtaining diffusivity data from gravimetric data. Diffusivity data was obtained by fitting the attenuation data to the expression provided by Stejskal and Tanner:

$$Ψ = exp [ - {( γ g δ )}^{2} ( Δ - \frac{δ}{3} ) ] \qquad ( 9 )$$

where ψ r is the signal attenuation, y is the gyromagnetic ratio of a proton, δ is the duration of the gradient pulse, g is the magnitude of the gradient pulse and Δ is the duration between two gradient pulses in the stimulated echo sequence. In this representative curve, δ=1.0 ms, Δ=10.0 ms and the gradient strength (g) is varied between 5.0 and 1000.0 G/cm.

Diffusion (Cont.). The M-S diffusivity of n-butanol is shown in FIG. 15. The n-butanol M-S diffusivity was measured by gravimetric uptake at 308° K. FIG. 15 shows that there is no appreciable change in water self-diffusivity for water as the linker composition is varied. This is due to the much smaller kinetic diameter of water (2.6 Å) in relation to the effective pore sizes of all the ZIF-8-90 materials. The diffusivity of methanol shows a small but systematic increase with decreasing ZIF-8 linker fraction. Ethanol shows an order-of-magnitude tunability of self-diffusivity and M-S diffusivity, whereas n-butanol shows over two orders-of-magnitude tunability of the M-S diffusivity as a function of the linker composition. As the size of the diffusing molecule increases, the effective pore size has a more pronounced effect on the diffusivity at a given linker composition, as well as the sensitivity of the diffusivity to changes in the linker composition. This behavior strongly corroborates the molecular sieving nature of the observed diffusion characteristics in the mixed-linker ZIF-8-90 series. The self-diffusivities of methanol and ethanol in pure ZIF-8 and ZIF-90 (see FIG. 15) are in good agreement with those measured previously by PFG-NMR.¹⁴ The ethanol diffusivity in ZIF-8, previously measured using infrared microscopy (IRM), also compares well with FIG. 15.

Summary In these examples, the inventors have demonstrated the continuous tuning of molecular sieving and adsorption behavior in mixed-linker ZIF-8-90 frameworks, which is due to the tunability of effective pore size as well as the ratio of polar and non-polar functional groups in the framework. These results are facilitated by the synthesis of a range of ZIF-8-90 mixed-linker materials with average crystal sizes spanning from 338 nm to almost 100 μm, and the detailed determination of the CSDs. Micro-Raman composition analysis of individual ZIF-8-90 crystals conclusively shows the hybrid nature and high uniformity of the mixed-linker materials. Tunable molecular sieving is observed both in non-polar alkanes as well in strongly polar alcohols, whereas tunable adsorption behavior is primarily observed for polar molecules like water and alcohols. The n-butane and i-butane diffusivities and the n-butane/i-butane diffusion selectivity can be continuously tuned over several orders of magnitude, allowing the selection of suitable materials for membrane-based separation of these isomers. Diffusion measurements of water and alcohols also reveal the strong dependence of tunable diffusivity on the molecular sizes and ZIF-8-90 pore sizes. The adsorption affinities of water and alcohols at low pressures are also strongly tunable by the variation of linker composition. This detailed demonstration of tunable adsorption and diffusion properties in ZIF-8-90 materials opens up the wider applicability of mixed-linker ZIF materials as a platform for a variety of membrane-based and adsorption -based molecular separations.

The embodiments and examples set forth herein are presented to best explain the present invention and its practical application and to thereby enable those skilled in the art to make and utilize the invention. However, those skilled in the art will recognize that the foregoing description and examples have been presented for the purpose of illustration and example only. The description as set forth is not intended to be exhaustive or to limit the invention to the precise form disclosed. Many modifications and variations are possible in light of the above teaching without departing from the spirit and scope of the following claims. The invention is specifically intended to be as broad as the claims below and their equivalents.

Definitions.

As used herein, the terms “a,” “an,” “the,” and “said” means one or more, unless the context dictates otherwise.

As used herein, the term “about” means the stated value plus or minus a margin of error or plus or minus 10% if no method of measurement is indicated. As used herein, the term “or” means “and/or” unless explicitly indicated to refer to alternatives only or if the alternatives are mutually exclusive.

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the terms “containing,” “contains,” and “contain” have the same open-ended meaning as “comprising,” “comprises,” and “comprise,” provided above.

As used herein, the terms “having,” “has,” and “have” have the same open-ended meaning as “comprising,” “comprises,” and “comprise,” provided above.

As used herein, the terms “including,” “includes,” and “include” have the same open-ended meaning as “comprising,” “comprises,” and “comprise,” provided above.

As used herein, the phrase “consisting of” is a closed transition term used to transition from a subject recited before the term to one or more material elements recited after the term, where the material element or elements listed after the transition term are the only material elements that make up the subject.

As used herein, the term “simultaneously” means occurring at the same time or about the same time, including concurrently.

Abbreviations. Abbreviations are used in this disclosure, as follows:

| b | Langmuir affinity constant |
|---|---|
| C | Absorbate concentration in |
| | sample |
| Cₛ | Langmuir capacity constant |
| CD₃COOD | D4-Acetic acid |
| CSD | Crystal size distribution |
| D | Transport diffusivity |
| Dₒ | Corrected M-S diffusivity |
| Dₜ | Transport diffusivity |
| δ | Duration of gradient pulse |
| Δ | Duration between two gradient |
| | pulses in simulated echo |
| | sequence |
| DI-H₂O | Deionized water |
| DMF | Dimethylformamide |
| DLS | Dynamic Light Scattering |
| FT-Raman | Fourier-Transform Raman |
| ΔHᵢₛₒ | Isosteric heat of adsorption |
| ¹H-NMR | Proton nuclear magnetic |
| | resonance |
| γ | Gyromagnetic ration of proton |
| IRM | Infrared microscopy |
| Λ | Fraction of adsobate absorbed by |
| | crystal |
| M | Moles |
| Mᵢ | Mass fraction of crystals |
| M-S diffusivity | Maxwell-Stefan diffusivity |
| 2-MeIm | 2-Methylimidazole |
| MeOH | Methanol |
| MOF | Metal-Organic Framework |
| NaCOOH | Sodium formate |
| N₂ | Nitrogen |
| NMR | Nuclear magnetic resonance |
| OHC-Im | Carboxyaldehyde-2-imidazole |
| PFG-NMR | Pulsed field gradient-NMR |
| R | Equivalent average radius of |
| | spherical sample |
| Rᵢ | Radius of crystal |
| RHO | Three-letter code signifying a |
| | particular type of zeolite |
| | structure topology as defined by |
| | International Zeolite Association |
| | (IZA) (see IZA website, |
| | available at |
| | http://izasc.fos.su.se/fmi/xsl/IZA- |
| | SC/ft.xsl) |
| ψ | Signal attenuation |
| SEM | Scanning Electron Microscope |
| SOD | Three-letter code signifying a |
| | particular type of zeolite |
| | structure topology as defined by |
| | International Zeolite Association |
| | (IZA) (see IZA website, |
| | available at |
| | http://izasc.fos.su.se/fmi/xsl/IZA- |
| | SC/ft.xsl) |
| T₂ | Relaxation constant |
| XRD | X-ray diffraction |
| Zn(NO₃)₂ | Zinc nitrate |
| ZIF | Zeolitic Imidazolate Framework |
| ZIF-8 | Zeolitic Imidazolate Framework- |
| | 8 |
| ZIF-8-90 | Zeolitic Imidazolate Framework- |
| | 8-90 |
| ZIF-90 | Zeolitic Imidazolate Framework- |
| | 90 |

Incorporation By Reference. All patents and patent applications, articles, reports, and other documents cited herein are fully incorporated by reference to the extent they are not inconsistent with this invention, as follows:
1. Cohen S M, Postsynthetic Methods for the Functionalization of Metal-Organic Frameworks, CHEM. REVS. 112(2) (2011) 970-1000.
2. Pan L, Olson D H, Ciemnolonski L R, Heddy R, Li J, Separation of Hydrocarbons with a Microporous Metal-Organic Framework, ANGEWANDIE CHEM1E 118(4) (2006) 632-635.
3. Getman R B, Bae Y-S, Wilmer C E, Snurr R Q, Review and analysis of molecular simulations of methane, hydrogen, and acetylene storage in metal-organic frameworks, CHEM. REVS. 112(2) (2011) 703-723.
4. Zhang C, Dai Y, Johnson J R, Karvan O, Koros W J, High Performance ZIF-8/6FDA-DAM Mixed Matrix Membrane for Propylene/Propane Separations, J. MEMBR. SCI. 389 (2012) 34-42.
5. Kwon H T, Jeong H-K, In Situ Synthesis of Thin Zeolitic-Imidazolate Framework ZIF-8 Membranes Exhibiting Exceptionally High Propylene/Propane Separation, J. Am. CHEM. SOC. 135(29) (2013) 10763-10768.
6. Dai Y, Johnson J R, Karvan O, Sholl D S, Koros W J, Ultem®/ZIF-8 mixed matrix hollow fiber membranes for CO₂/N₂ separations, J. MEMBR. SCI. 401-402 (2014) 76-82.
7. Pan Y, Lai Z, Sharp separation of C2/C3 hydrocarbon mixtures by zeolitic imidazolate framework-8 (ZIF-8) membranes synthesized in aqueous solutions, CHEM. COMM'NS 47 (2011) 10275-10277.
8. Phan A, Doonan C J, Uribe-Romo F J, Knobler C B, O′Keeffe M, Yaghi O M, Synthesis, Structure, and Carbon Dioxide Capture Properties of Zeolitic Imidazolate Frameworks, ACCOUNTS OF CHEMICAL RESEARCH 43(1) (2009) 58-67.
9. Banerjee R, Phan A, Wang B, Knobler C B, Furukawa H, O′Keeffe M, et al., High-Throughput Synthesis of Zeolitic Imidazolate Frameworks and Application to CO₂ Capture, SCIENCE 319 (2008) 939-943.
10. Park K S, Ni Z, Cote A P, Choi J Y, Huang R, Uribe-Romo F J, et al., Exceptional Chemical and Thermal Stability of Zeolitic Imidazolate Frameworks, Proceedings of the National Academy of Sciences of the United States of America 103(27) (2006) 10186-10191.
11. Li J-R, Kuppler R J, Zhou H-C, Selective gas adsorption and separation in metal-organic frameworks, CHEM. SOC. REVS. 38 (2009) 1477-1504.
12. Li J-R, Ma Y, McCarthy M C, Sculley J, Yu J, Jeong H-K, et al., Carbon dioxide capture-related gas adsorption and separation in metal-organic frameworks, COORD. CHEM. REVS. 255 (2011) 1791-1823.
13. Thompson J A, Blad C R, Brunelli N A, Lydon M E, Lively RP, Jones C W, et al., Hybrid Zeolitic Imidazolate Frameworks: Controlling Framework Porosity and Functionality by Mixed-Linker Synthesis, CHEM. MATERS. 24(10) (2012) 1930-1936.
14. Gee J A, Chung J, Nair S, Sholl D S, Adsorption and Diffusion of Small Alcohols in Zeolitic Imidazolate Frameworks ZIF-8 and ZIF-90, J. PHY. CHEM. C 117 (2013) 3169-3176.
15. Zhang K, Lively R P, Dose M E, Brown A J, Zhang C, Chung J, et al., Alcohol and water adsorption in zeolitic imidazolate frameworks, CHEM. COMM'NS 49 (2013) 3245.
16. Zhang K, Lively R P, Zhang C, Koros W J, Chance R R, Investigating the Intrinsic Ethanol/Water Separation Capability of ZIF-8: An Adsorption and Diffusion Study, J. PHY. CHEM. C 117 (2013) 7214-7225.
17. Thompson J A, Brunelli N A, Lively R P, Johnson J R, Jones C W, Nair S, Tunable CO2 Adsorbents by Mixed-Linker Synthesis and Postsynthetic Modification of Zeolitic Imidazolate Frameworks, J. PHY. CHEM. C 117(16) (2013) 8198-8207.
18. Cravillon J, Schroder C A, Bux H, Rothkirch A, Caro J, Wiebcke M, Formate modulated solvothermal synthesis of ZIF-8 investigated using time-resolved in situ X-ray diffraction and scanning electron microscopy, CRYSTENGCOMM 14(2) (2012) 492-498.
19. Cravillon J, Münzer S, Lohmeier S-J, Feldhoff A, Huber K, Wiebcke M, Rapid Room-Temperature Synthesis and Characterization of Nanocrystals of a Prototypical Zeolitic Imidazolate Framework, CHEM. MATERS. 21(8) (2009) 1410-1412.
20. Kong X, Deng H, Yan F, Kim J, Swisher J A, Smith B, et al., Mapping of Functional Groups in Metal-Organic Frameworks, SCIENCE 341 (2013) 882-885.
21. Zhang K, Zhang L, Jiang J, Adsorption of C1-C4 Alcohols in Zeolitic Imidazolate Framework-8: Effects of Force Fields, Atomic Charges and Framework Flexibility, J. PHY. CHEM. C 117 (2013) 25628-25635.
22. Fairen-Jimenez D, Moggach S A, Wharmby M T, Wright P A, Parsons S, Duren T. Opening the Gate: Framework Flexibility in ZIF-8 Explored by Experiments and Simulations, J. AM. CHEM. SOC. 133(23) (2011) 8900-8902.

What is claimed is:

1. A method for forming a hybrid zeolitic imidazolate framework (ZIF) comprising the formation steps of:
a. preparing a first solution comprising:
i. a 2-methylimidazolate or a functionalized derivative thereof; and
ii. a carboxaldehyde-2-imidazolate or a functionalized derivative thereof;
b. preparing a second solution comprising a metal ion; and
c. combining the first solution and the second solution to form the hybrid ZIF, wherein a first fraction of 2-methylimidazolate or a functionalized derivative thereof in the hybrid ZIF is from about 5 to about 95 or any value there between and a second fraction carboxaldehyde-2-imidazolate or a functionalized derivative thereof in the hybrid ZIF is 100—the first fraction.

2. The method of claim 1, wherein the first fraction is from about 55 to about 70 or any value there between.

3. The method of claim 1, the first fraction is from about 25 to about 35 or any value there between.

4. The method of claim 1, wherein the first fraction is from about 5 to about 10 or any value there between.

5. The method of claim 1, wherein the metal ion comprises a transition metal.

6. The method of claim 1, wherein the metal ion comprises zinc.

7. The method of claim 1, wherein the metal ion comprises cobalt.

8. The MOF of claim 1, wherein the hybrid ZIF has a n-butane/i-butane diffusion selectivity of at least 1.2 times greater than a non-hybrid ZIF-90.

9. The MOF of claim 1, wherein the hybrid ZIF has a n-butane/i-butane diffusion selectivity of at least 5.7 times greater than a non-hybrid ZIF-90.

10. The MOF of claim 1, wherein the hybrid ZIF has a n-butane/i-butane diffusion selectivity of at least 40 times greater than a non-hybrid ZIF-90.

11. The MOF of claim 1, wherein the hybrid ZIF has a butanol/water adsorption selectivity of at least 1.2 times greater than a non-hybrid ZIF-8.

12. The MOF of claim 1, wherein the hybrid ZIF has a butanol/water adsorption selectivity of at least 5 times greater than a non-hybrid ZIF-8.

13. The MOF of claim 1, wherein the hybrid ZIF has a butanol/water adsorption selectivity of at least 10 times greater than a non-hybrid ZIF-8.

14. The method of claim 1, further comprising an activation step to remove impurities from the hybrid ZIF.

15. The method of claim 14, wherein the activation step comprises heat treating or vacuum degassing from about 100° C. to about 300° C.

16. The method of claim 1, further comprising reaction step to functionalize the hybrid ZIF.

17. The method of claim 16, wherein the reaction step comprises exposing the hybrid ZIF to a reactive agent.

18. The method of claim 17, wherein the reactive agent comprises functionality selected from the group consisting of alkyl, amino, chloro, bromo, carbonyl, nitro, sulfonate, hydroxy, hydroxo, aldehyde, organometallic functionalities and combinations thereof.

19. The method of claim 17, wherein the reactive agent is aldehyde.

20. A metal-organic framework (MOF) comprising:
a. a hybrid zeolitic imidazolate framework (ZIF) of claim 1 comprising:
i. a 2-methylimidazolate, wherein the first fraction of 2-methylimidazolate in the hybrid ZIF is from about 5 to about 70 or any value there between;
ii. a carboxaldehyde-2-imidazolate, wherein the second fraction of carboxaldehyde-2-imidazolate in the hybrid ZIF is 100—the first fraction; and
iii. a metal ion.

21. The MOF of claim 20, wherein the first fraction is from about 55 to about 70 and any value there between.

22. The MOF of claim 20, the first fraction is from about 25 to about 35 and any value there between.

23. The MOF of claim 20, wherein the first fraction is from about 5 to about 10 and any value there between.

24. The MOF of claim 20, wherein the metal ion comprises a transition metal.

25. The MOF of claim 20, wherein the metal ion comprises zinc.

26. The MOF of claim 20, wherein the metal ion comprises cobalt.

27. The MOF of claim 20, wherein the hybrid ZIF has a n-butane/i-butane diffusion selectivity of at least 1.2 times greater than a non-hybrid ZIF-90.

28. The MOF of claim 20, wherein the hybrid ZIF has a n-butane/i-butane diffusion selectivity of at least 5.7 times greater than a non-hybrid ZIF-90.

29. The MOF of claim 20, wherein the hybrid ZIF has a n-butane/i-butane diffusion selectivity of at least 40 times greater than a non-hybrid ZIF-90.

30. The MOF of claim 20, wherein the hybrid ZIF has a butanol/water adsorption selectivity of at least 1.2 times greater than a non-hybrid ZIF-8.

31. The MOF of claim 20, wherein the hybrid ZIF has a butanol/water adsorption selectivity of at least 5 times greater than a non-hybrid ZIF-8.

32. The MOF of claim 20, wherein the hybrid ZIF has a butanol/water adsorption selectivity of at least 10 times greater than a non-hybrid ZIF-8.

33. The MOF of claim 20, further comprising a functionalized hybrid ZIF.

34. The MOF of claim 33, wherein the functionalized hybrid ZIF comprises an aldehyde.

35. The MOF of claim 33, wherein the functionalized hybrid ZIF comprises an amine.

36. A molecular sieve device comprising metal-organic framework (MOF) comprising:
a. a hybrid zeolitic imidazolate framework (ZIF) of claim 1 comprising:
i. a 2-methylimidazolate or a functionalized derivative thereof, wherein the first fraction of the 2-methylimidazolate or the functionalized derivative thereof in the hybrid ZIF is from about 5 to about 90 or any value there between;
ii. a carboxaldehyde-2-imidazolate or a functionalized derivative thereof, wherein the second fraction the carboxaldehyde-2-imidazolate or the functionalized derivative thereof in the hybrid ZIF is 100—the first fraction; and
iii. a metal ion.

37. The device of claim 36, wherein the first fraction is from about 55 to about 70 and any value there between.

38. The device of claim 36, wherein the first fraction is from about 25 to about 35 and any value there between.

39. The device of claim 36, wherein the first fraction is from about 5 to about 10 and any value there between.

40. The device of claim 36, wherein the hybrid ZIF has a n-butane/i-butane diffusion selectivity of at least 1.2 times greater than a non-hybrid ZIF-90.

41. The device of claim 36, wherein the hybrid ZIF has a n-butane/i-butane diffusion selectivity of at least 5.7 times greater than a non-hybrid ZIF-90.

42. The device of claim 36, wherein the hybrid ZIF has a n-butane/i-butane diffusion selectivity of at least 40 times greater than a non-hybrid ZIF-90.

43. The device of claim 36, wherein the hybrid ZIF has a butanol/water absorption diffusion selectivity of at least 1.2 times greater than a non-hybrid ZIF-8.

44. The device of claim 36, wherein the hybrid ZIF has a butanol/water absorption selectivity of at least 5 times greater than a non-hybrid ZIF-8.

45. The device of claim 36, wherein the hybrid ZIF has a butanol/water absorption selectivity of at least 10 times greater than a non-hybrid ZIF-8.

46. The device of claim 36, wherein a feed composition to the device comprises about 2 mol % to about 95 mol % i-butane, about 2 mol % to about 95 mol % n-butane and mixtures thereof.

47. The device of claim 36, wherein the device is operated at a temperature from about 35° C. to about 95° C. or any value there between.

48. The device of claim 46, wherein the device is operated at a feed pressure from about 1 bar to about 14 bar or any value there between.

49. The device of claim 36, wherein a feed composition to the device comprises about 0 mol % to about 5 mol % methanol, about 0 mol % to about 5 mol % ethanol, about 0 mol % to about 5 mol % propanol, about 0 mol % to about 5 mol % butanol, about 2 mol % to about 95 mol % water and mixtures thereof.

50. The device of claim 49, wherein the device is operated at a feed pressure from about 1 bar to about 2 bar or any value there between.
